(19) United States
(12) Patent Application Publication
(10) Pub. No.: US 2024/0011977 A1
(43) Pub. Date: Jan. 11, 2024

(54) TISSUE PROCESSING

(71) Applicant: APPLIKATE TECHNOLOGIES, INC., Washington, DC (US)

(72) Inventors: Michael Levene, Washington, DC (US); Richard Torres, Washington, DC (US)

(21) Appl. No.: 18/347,348

(22) Filed: Jul. 5, 2023

Related U.S. Application Data
(60) Provisional application No. 63/358,367, filed on Jul. 5, 2022.

(51) Int. Cl. G01N 33/53; G01N 33/68; G01N 1/30; G01N 33/52

(52) U.S. Cl. CPC G01N 33/5308; G01N 33/6803; G01N 1/30; G01N 33/52; G01N 2001/302

(57) ABSTRACT

The present invention relates to systems and methods for tissue processing and imaging including simultaneous dehydration and staining with fluorescent nuclear and protein dyes followed by optical sectioning microscopy.

### FIELD OF THE INVENTION

The present disclosure relates to tissue processing and imaging systems and methods.

### BACKGROUND

Histology and histopathology involve the study of cells and tissues under a microscope to diagnose and monitor diseases, such as cancer. Differentially staining and imaging various structures in tissue can provide detailed information at the cellular and sub-cellular level that is indispensable in tissue analysis including diagnostic and prognostic evaluation.

While much of the field still depends on visual examination by trained medical professionals, the development of new processing and imaging techniques has increased throughput and imaging quality on the front end. For example, treatment with fluorescent dyes and the use of confocal microscopy and multiphoton microscopy provide high-quality images with good cellular detail at various depths in tissue. The ability to stain and image structures at depth in tissue allows for three-dimensional analysis of tissue samples which improves diagnostic accuracy. In contrast to diagnostic evaluation using visual inspection of physical thin sections of tissue, new techniques are intrinsically digital, enabling easy consultation, remote evaluation by specialist pathologists, and application of digital image analysis tools.

Fluorescent optical sectioning microscopy has not been extensively applied to high throughput environments. In particular, processing techniques for optical sectioning microscopy available to date are not amenable to automation, time efficiency, and cost control. They have also not been tuned to permit routine analysis by pathologists in the full range of clinically relevant tissues. As such, there exists a need for additional broadly-applicable, cost-effective, and time-efficient techniques to enable the application of optical sectioning microscopy in environments where efficiency is critical.

Additionally, short-pulse, high-intensity laser light can be used to generate mapping of second-harmonic generation (SHG), which is useful in imaging protein structures such as collagen and amyloid. Collagen imaging in liver can be used to quantify fibrosis, a marker of liver injury in a variety of hepatic conditions and an important clinical prognostic factor.

The evaluation of collagen fibrosis is a routine part of many types of histologic assessment owing to a recognized association with unrecoverable loss in function in various tissue types. Evaluation of collagen is routinely implemented for evaluation of fibrosis in kidney, liver and lung disease as a critical determinant of function and prognosis, as well as for delineation of critical structures in a variety of other tissues such as gastrointestinal tract, skin, breast, etc. Typically, specific stains are employed to highlight collagen presence in physical thin cut sections. This is done because other tissue features may be largely indistinguishable in the standard hematoxylin and eosin (H&E) stain. For example, smooth muscle may appear similar to collagen in standard H&E staining and be mischaracterized as fibrosis from collagen, leading to confusion and even misdiagnoses.

One such stain used specifically for characterization of fibrosis is called “trichrome”. Several versions of the trichrome stain exist but all involve multiple dyes, typically aiming to achieve contrast for recognition of collagen structures by staining collagen selectively, often by controlling the pH at staining in sequential steps, or by using dyes with varying selectivity for acidic or basic structures. Other techniques have been developed that involve antibodies against collagen (immunohistochemistry, IHC) or direct chemical stains such as Sirius Red which can be visualized under polarization light conditions to highlight with improved contrast the location of specific collagen types.

While these techniques represent significant advancement in the field of histology and histopathology, scaling up their use for every-day clinical application requires streamlining the processes to reduce costs, increase throughput, and provide consistent results. Many of the dyes used can be expensive and represent a significant portion of the reagent cost in the above techniques. Furthermore, dye concentrations are not standardized and providing consistent quality when imaging at depth can be problematic.

Furthermore, it is somewhat common practice to store tissue after formaldehyde fixation in a solution of 70% ethanol in water. This is typically done to arrest the formaldehyde cross-linking and preserve the tissue for an indefinite time in a relatively inert medium which resists microbial growth, thereby preventing tissue degradation. Through experimentation, we have noted that the use of ethanol solution for tissue storage has a detrimental effect on the binding of certain nucleic acid dyes, specifically fluorescent ones. Some literature suggests that ethanol complexes with nucleic acids in a manner which cannot be rapidly reversed through exposure to a methanol-based solution. As a result, alternations to a process with direct transfer of a specimen from ethanol solution to methanol based staining are needed to enable optimal staining in this setting.

### SUMMARY

Systems and methods for processing tissue samples are described. The systems and methods described herein allow for rapid and efficient processing and imaging of tissue with high resolution. Methods include the steps of obtaining a tissue sample and contacting the tissue sample with a fixative solution comprising at least one fixative and at least one fluorescent dye. In one embodiment, the method further includes the step of contacting the tissue sample with a clearing solution. In another embodiment, the method further includes the step of imaging the tissue sample. In another embodiment, the at least one fluorescent dye is selected from the group consisting of eosin, 4′,6-Diamidino-2-Phenylindole (DAPI), the nucleic acid stain sold under the trademark SYTOX green by ThermoFisher Scientific (Waltham, Massachusetts), the compound sold under the name Alexa Fluor 594-NETS by Molecular Probes acridine orange, rhodamine B, propidium iodide, and a Hoechst dye. In certain methods, samples may be simultaneously dyed with both protein and nuclear dyes. The nuclear dye, especially DAPI, may be used at much higher concentrations than previously recognized with surprising results in concert with high-speed imaging. For example, DAPI concentrations of about 50, about 100, about 200, about 300, about 400, about 500, about 600, about 700, about 800, or more micromolar (e.g., in many cases or more than reported in U.S. patent application Ser. Nos. 14/324,019 and 14/790,917, the contents of each of which are incorporated herein by reference) may be used. In preferred embodiments, DAPI concentration may be between 300 and 800 or 300 and 600 micromolar and more preferably around 400 micromolar.

In certain embodiments, the at least one fixative may be methacarn, while in some embodiments, formalin may be used as a fixation step and tissue processing may be completed without the use of chloroform as chloroform may contribute to reduced quality (e.g., speed and degree) of staining, particularly with DAPI. In another embodiment, the fixative solution further comprises a permeation enhancer such as acetic acid.

Acetic acid can be used to counteract tissue shrinkage and enhance fluorescent dye performance, especially with the combined nuclear and protein dyes used in various methods of the invention. However, increasing the hydrogen ionization potential beyond a certain value may, for example, reduce eosin fluorescence beyond a desirable level for adequate recognition of cellular features during laser imaging and/or place the tissue sample at risk to degradation and, as such, in certain embodiments, a pKa range (at room temperature) of between 4.82 and 5.2 and, preferably, between 4.86 and 5 may be maintained for formulations of the invention.

In another embodiment, the step of contacting the tissue sample with a fixative solution is performed at between about 40° C. and about 47° C. and preferably at about 45° C. In certain embodiments, the fixative solution further comprises a red blood cell lysing agent. In some embodiments, the step of contacting the tissue sample with a fixative solution is performed over a period of time of about 1 hour. In another embodiment, the step of contacting the tissue sample with a fixative solution is performed over a period of less than 15 minutes. In another embodiment, the clearing solution comprises benzyl alcohol and benzyl benzoate. In another embodiment, the ratio of benzyl alcohol to benzyl benzoate is about 1:2. In another embodiment, the step of contacting the tissue sample with a clearing solution is performed over a period of time of about 10 minutes. In another embodiment, a partially fixed and a partially cleared tissue is placed in fixative after imaging. In another embodiment, the steps of contacting the tissue sample with a fixative solution and contacting the tissue sample with a clearing solution are performed over a period of time of about 1.5 hours. In other embodiments, the steps take place over a period of about 4 hours. In other embodiments, the time period is about 8 hours, matching established incubations periods in existing tissue processing protocols, but with the incorporation of the unique reagent mixtures described herein. In another embodiment, the tissue sample has been fixed prior to obtaining the tissue sample. In some embodiments, the tissue has been previously fixed in formalin. In some embodiments, the tissue has been stored in 70% ethanol.

In certain embodiments, a combined dehydrating/staining solution for use in tissue processing may include methanol as a dehydrant and fluorescent dyes including DAPI at a concentration between about 300 μM and about 800 μM and eosin in a concentration between about 0.025 nM and about 2.5 nM with the solution having a pKa between about 4.84 and about 5. The solution may include about 10% acetic acid to achieve the desired pKa. Such dehydrating/staining solutions may have a reduced shelf life of about 3 days before staining quality and/or speed will be noticeably reduced when stored at room temperature. However, methods of the invention may include storing the solution at low temperatures or about −20° C. or lower (e.g., about −80° C.) in order to significantly increase shelf life and reduce degradation of stains in the dehydrating/staining solution.

The tissue sample may be fixed in formalin prior to exposure to the dehydrating/staining solution. The tissue sample may be incubated in formalin for about 5 minutes or more. The tissue sample may be treated with the dehydration/staining solution at about between about 40° C. and about 47° C.

In some embodiments, the tissue has been placed in ethanol solution prior to obtaining the tissue samples. In this scenario, the inhibition of nucleic acid dye binding by ethanol can be overcome by removing the ethanol prior to dyeing. The tissue can be placed in aqueous solution such as water, saline, or buffered saline, or formalin solution, for a period of time that is proportional to tissue thickness, thereby ensuring adequate penetration of the water to displace the ethanol that may be interacting with nucleic acids. Similarly, in other embodiments methanol devoid of nucleic acid dye can be used to displace the ethanol. The time for ethanol removal can be of the order of 10 minutes for specimens a few hundred microns thick, or for thicker specimens when the goal is to image at no more than a few hundred microns. Deeper imaging of thicker specimens requires longer exposure to the aqueous solution which can take place over 30 minutes, 1 hour, 2 hours, 4 hours, 8 hours, 16 hours, or 24 hours.

Also described is a method of imaging a tissue sample. The method includes the steps of obtaining a tissue sample, contacting the tissue sample with a fixative solution comprising at least one fluorescent dye, contacting the tissue sample with a clearing solution, and producing a tissue sample image by measuring intensity values of the fluorescence of the tissue sample, and, optionally, converting the intensity values to effective optical densities, such that the optical densities recreate the coloration of a stain in a produced image of the tissue sample. In one embodiment, the tissue sample image is produced using an optical sectioning microscope. In various embodiments, the optical sectioning microscope is selected from the group consisting of: a multiphoton microscope (MPM), a confocal microscope, a structured illumination microscope, a super-resolution microscope, a selective plane illumination microscope (SPIM), a side-plane illumination microscope, a spinning disk confocal microscope, and a deconvolution microscope. In another embodiment, the step of producing a tissue sample image further comprises second harmonic generation (SHG). In another embodiment, the sample image is a three-dimensional (3-D) sample image. In another embodiment, the sample image is obtained at a sample depth of greater than 50 μm. In another embodiment, the intensity values are converted to effective optical densities using an exponential pseudo-coloration process.

In certain embodiments, the ability of eosin and other protein stains to fluoresce can be used in pre-imaging analysis in order to identify target regions for subsequent imaging. This has the advantage of producing high contrast images that are specific to tissue, as an aid in identifying tissue position. For example, methods may include taking a standard image (non-microscopic) of a sample stained with eosin or another stain that fluoresces. Images may be taken with specific wavelength excitation such as red light, or green light, or white light, or with filters, lasers, or LEDs that correspond to such wavelength ranges, and optionally collected using filters that isolate specific wavelengths. The image(s) can then be processed by isolating relevant signal by wavelengths of emission, such as green wavelengths (for eosin) or orange/red wavelengths (for Alexa-594 or Atto-594). From this analysis, it is possible to create a bounding box around the region corresponding to tissue and restrict image collection to that area. Accordingly, microscopic imaging of space devoid of tissue can be avoided allowing for more efficient use of the imaging time.

Certain systems and methods of the invention may include “macro” or microscopic imaging data (e.g., pre or post-processing size, shape, or features) of a substantially immobilized tissue sample as a digital fingerprint associated with patient, laboratory, or other identification information. By “macro” what is meant is any image of the tissue specimen obtained through the use of photographic cameras such as those that employ charge coupled devices (CCDs) or complementary metal oxide semiconductors (CMOS) or similar devices. Accordingly, even in the absence of standard labels or barcodes on a physical element (such as if the identifier of a slide or a tissue block or cassette is removed or damaged in some manner), a tissue sample can be subsequently identified using, for example, an image comparative algorithm. This approach is applicable to any prepared tissue-sample image and is particularly useful for identifying physical tissue microscope slides as well as samples prepared for imaging using optical sectioning microscopy such as for confocal, multiphoton, light-sheet, deconvolution, or super-resolution microscopy. The image used for identification may be obtained in any of various ways: it may be a microscopic image of a specimen or may be a photograph using standard white light illumination and standard red, green, and blue sensor camera detection, or it may employ the use of specific illumination and detection filters as described herein for improving the isolation and characterization of the relevant region of interest of biological material.

In various embodiments, treatment with the dehydrating/staining solutions of the invention can be used to produce a visualization of tissues containing acid polysaccharides. Subsequent imaging methods can include converting two channel fluorescence to H&E-like coloration resulting in an image that appears similar to a combination of H&E and Alcian Blue staining. While this combination is not typically performed with physical slides, it offers an opportunity to perform a more complete assessment of tissue, including both standard morphologic assessment and ready quantitation of mucin, with fewer staining steps, while not adversely affecting standard H&E assessment.

The present invention further provides tissue processing techniques using tissue-specific fluorescent dye concentrations for achieving increased image quality and contrast at depth along with reduced waste as compared to existing techniques. Systems and methods of the invention recognize that imaging quality at depth is dependent on tissue type and associated protein content. Despite a logical assumption that imaging of samples with higher protein content would benefit from higher fluorescent protein dye concentration, the inventive methods observe that image quality for such samples actually benefits from lower concentration of fluorescent protein dye during specimen preparation.

Practically applying those principles, methods of the invention include assessing tissue samples for protein content and varying the protein dye concentration used in staining based on that protein content. The tissue-specific protein dye concentrations are generally higher for tissues having lower protein content (e.g., renal tissue) than in tissues with higher protein content (e.g., epithelial tissue). Relatively higher concentrations result in improved contrast at depth in samples with relatively low protein content. However, similar concentrations of protein dyes in samples with higher protein content result in increased signal absorption from shallower levels of the sample, decreasing contrast and degrading image quality in optical sectioning microscopy methods when imaging deeper levels within the sample (e.g., confocal imaging, multiphoton imaging, and selective plane illumination). Increased absorption can affect both nuclear and protein fluorescent signals based on the relative emission and absorption spectra of the specific dyes employed. However, due to the overall higher concentration of protein than nucleic acids in tissue, the interplay between protein-sensitive dye concentration and protein content has a significantly greater impact on image quality at depth.

An additional benefit of tailoring the dye concentration to the protein content is the efficient use of expensive reagents. Counterintuitively using less dye to stain tissue with higher protein content results in cost savings that can help promote the fluorescent imaging techniques described above for every-day clinical use.

The increased signal absorption resulting from excessive dye concentration in protein-rich samples can also affect the ability to detect second harmonic generation at depth. As noted above, SHG detection is highly relevant for fibrosis detection in kidney and liver samples. Accordingly, tailoring dye concentration to the protein content of a sample also supports more accurate SHG detection and the related diagnostic and prognostic evaluations afforded by SHG detection in certain tissues.

Combined protein and nucleic acid staining can be used to accurately reproduce common histologic stains for pathologist interpretation such as the Hematoxylin and Eosin stain (H&E) and the so-called Trichrome stain. In such applications, with the benefit of normalization of fluorescent signal intensity after signal acquisition and the adaptive nature of back-end visual analysis, the use of the lower concentrations of protein-specific fluorescent dyes in high protein density samples still yields images of sufficient quality to replicate standard H&E and Trichrome staining.

Aspects of the invention include a method of imaging a tissue sample by determining a protein dye concentration for a given tissue type based on the protein density or content of that tissue. The protein dye concentration is inversely related to the protein density. A tissue sample of the tissue type is then contacted with a solution comprising at least one fluorescent protein dye at the determined concentration for that tissue type. A tissue sample image can then be produced by measuring intensity values of fluorescence of the tissue sample at a depth greater than about 200 μm. The solution may include a fluorescent nuclear dye which may be selected from DAPI, SYTOX dyes, SYTO dyes, propidium iodide, acridine orange, or Hoechst dyes.

The at least one fluorescent protein dye may include eosin, Rhodamine B (RhB), Alexa 594 NHS-ester and equivalents, or ANS. Producing the tissue sample image can include second harmonic generation (SHG). In certain embodiments, the tissue sample may include collagen. A typical example of alcoholic stock solution of eosin is Eosin Y, 1% alcoholic solution, non-acidic (Polysciences, Inc, Warrington PA), or any solution composed of 1% weight eosin by volume. In some embodiments, the alcoholic stock solution is eosin as 1% eosin weight by volume in methanol. In other embodiments, the stock solution is 1% eosin weight by volume in dimethyl sulfoxide (DMSO). The tissue sample may be derived from any biological source and the at least one fluorescent protein dye may include eosin at about 0.4% of stock solution. In some embodiments, the tissue sample can be a liver sample and the at least one fluorescent protein dye may include eosin at about 0.02% of stock solution. In certain embodiments, a tissue sample may be cleared prior to exposure to a nuclear fluorescent dye and then irradiated with pulsed laser light to produce an axially sectioned image of SHG molecules (e.g., collagen) to avoid interference from eosin or other fluorescent stains while providing valuable information regarding collagen composition in the tissue sample.

In various embodiments, the tissue sample may be renal tissue and the at least one fluorescent protein dye can include eosin at about 0.4% of stock solution. Alternatively, the tissue sample can be epithelial tissue and the at least one fluorescent protein dye may include eosin at about 0.04% of alcoholic stock solution. The sample image can be a three dimensional (3-D) sample image. The tissue sample image can be produced using an optical sectioning microscope. The optical sectioning microscope may be a multiphoton microscope (MPM), a confocal microscope, a structured illumination microscope, a super-resolution microscope, a selective plane illumination microscope (SPIM), a side-plane illumination microscope, a spinning disk confocal microscope, or a deconvolution microscope.

The solution may further comprise a fixative such as methacarn. Methods of the invention may include contacting the tissue sample with a clearing solution before producing the tissue sample image. The clearing solution may include benzyl alcohol and benzyl benzoate. The solution can include a permeation enhancer. In certain embodiments methods of the invention may include converting the intensity values to effective optical densities, such that the optical densities recreate the coloration of a stain in the tissue sample image.

### BRIEF DESCRIPTION OF THE DRAWINGS

FIG. 1 shows mucin staining with dehydrating/staining solutions according to various embodiments.

### DETAILED DESCRIPTION

The present invention relates to methods of tissue preparation and image-analysis that allow for the practical implementation of the deep imaging of tissue specimens. The methods described herein reduce the number of steps for tissue processing, decrease the time required to process tissues, and improve the clarity and contrast in samples, thereby permitting deep tissue imaging of the sample. As demonstrated herein, the methods of the present invention provide complete visualization of biopsy-sized specimens without the need for the time-consuming and manually intensive post-clearing steps, thereby reducing the time between biopsy through morphologic assessment. In non-limiting examples, cleared biopsy specimens can be provided to pathologists for direct visualization or scanned for image distribution. In another non-limiting example, a primary diagnosis may be rendered based on the images, with subsequent studies ordered if necessary. In another non-limiting example, specimens can be partially fixed and partially cleared for intra-operative evaluation or for any other clinical scenario where a fast visual examination is desired. In further examples, partially fixed specimens may be fully fixed at a later time and partially cleared specimens may be fully cleared at a later time.

As described in U.S. patent application Ser. Nos. 14/324,019 and 14/790,917 and discussed in more detail below, methods for deep imaging of tissue samples simultaneously stained with both nucleic acid-specific and protein-specific dyes have been previously reported. Such techniques have been applied to reproduce common histologic stains for pathologist interpretation such as H&E and Trichrome stains. In certain embodiments, methods of the invention recognize an inverse relationship between protein content in a tissue sample and the protein dye concentration required to provide suitable images at depth. Certain systems and methods of the invention recognize that imaging quality at depth is dependent on tissue type where, counterintuitively, imaging of samples with higher protein content benefits from lower concentration of fluorescent protein dye, especially certain protein fluorescent dyes such as eosin, during specimen preparation. Meanwhile, samples with relatively low protein content exhibit improved contrast at depth when stained with high concentrations of fluorescent protein dyes such as eosin.

Relative to the methods disclosed in U.S. patent application Ser. Nos. 14/324,019, 14/790,917, and 17/000,994, the content of each of which is incorporated herein, systems and methods herein can extend the capability to ensure that cells which are particularly susceptible to damage from certain processing reagents are able to remain microscopically intact. In certain embodiments, dehydrating/staining formulations are described which allow improved signal at depth in a broader range of samples. In certain embodiments, the improvements of processing and imaging methods are ensured by minimizing the use of formaldehyde fixation, itself a function of time, tissue thickness, and temperature, as well as modifying the staining formulation by eliminating one component while controlling the pKa to within a relatively narrow range. Elimination of a previously disclosed component of the dehydration/staining solution, chloroform, results from better understanding of the role it plays and the reduced necessity for it with adequate formalin fixation combined with the use of a specific pKa range.

### Tissue Fixing and Chloroform-Free Treatment Methods

In some forms of traditional tissue processing, methanol acts as a dehydrant that replaces tissue water and, due to its miscibility with organic solvents such as xylene, acts as an intermediary between these steps, the latter of which then allows the infiltration with molten paraffin wax. In other words, methanol is a first step in the goal of replacing tissue water with paraffin wax in current practice. Methanol also can act as a fixative itself. Fixation is the process by which enzymatic activity of live cells, which would lead to cellular degradation, is arrested. Formaldehyde arrests enzymatic activity by a chemical reaction that leads to cross-linking of proteins, including enzymes, arresting their degradation activity. Methanol, and other alcohols, can achieve the same goal by merely replacing the water. Water is needed by enzymes to function and the alcohol dehydration results in arrest of enzymatic activity. Hence, methanol itself can function as a “fixative”.

In earlier methods, processing times were reduced by placing tissue directly in methacarn. It has been noted that red blood cells treated according to earlier methods were prone to lysing (breaking apart), more so than other cell types. Without wishing to be tied to a particular theory, it is believed that the acid exposure of red cells that have not had their membranes stabilized by the cross-linking offered by formalin causes them to swell and explode. In certain embodiments such lysis can be a potential benefit, understanding that the absorption of both excitation laser light and emitted fluorescence by red cells could be diminished, improving the imaging of the more relevant cells.

Further investigation has revealed that certain other cell types, such as renal tubular epithelial cells, are also extremely sensitive to direct exposure to methacarn. Damage to those other cell types negatively affects histology interpretation, so it is desirable to avoid such direct exposure in certain embodiments and with certain tissue types. In such circumstances, it is beneficial to perform some degree of formalin fixation (e.g., in buffered solution at neutral pH) prior to exposure of the tissue to an acid-containing dehydrant such as those described herein.

A formaldehyde solution or formalin fixation step can be generally employed for any tissue type, though there are tissues that do not require it. Kidney morphology preservation can benefit from formalin exposure (standard 10% formaldehyde formulation). Liver can benefit from it also. Prostate, breast, and lung do not usually necessarily receive the same advantages but do not necessarily suffer from such exposures and, as such, a standardized warm formalin fixation prior to combined stain/dehydration works in those tissues as well. In certain embodiments, it may be advantageous to reduce or eliminate formalin exposure in order to avoid degradation of nucleic acids for subsequent DNA and RNA analysis. Accordingly, tissue type (as discussed above) as well as desired preference for morphology or nucleic acid analysis may be considered in determining whether or not to include a formalin or other fixative exposure step prior to dehydration/staining. Because, as discussed above, alcohol dehydration such as methacarn exposure can be used to fix tissue, use of formaldehyde fixation prior to such acidic alcohol dehydration steps are not historically used and the advantages of such a step, recognized herein, are a contribution of the present application.

In various embodiments a key modification to prior methods is the elimination of chloroform. Chloroform is variably reported as playing two roles in methacarn fixation. One is as a morphology preservative, which refers to the counteracting of tissue shrinkage that occurs during dehydration. The other is potentially as an aid in rapid tissue penetration owing to its low viscosity and dehydrating effect. This latter effect is likely minimal relative to methanol since methanol also has a very low viscosity. Certain methods of the present invention recognize that inclusion of chloroform in the dehydrations/dual staining processing steps can reduce the quality of staining, particularly the speed and degree of nuclear staining using DAPI. Another contribution of the present invention is a surprising finding that the morphology-preserving effect of chloroform is of little to no significance in pathology applications when optical sectioning microscopy is used. While some evidence of tissue shrinkage has been observed, the degree of collapse that occurs is not detectable on routine microscopic assessment of the images. It is possible that the acid itself has sufficient shrinkage counteracting effects. Formalin fixation prior to dehydration/staining may also lend sufficient structure to the cells to reduce or avert cell shrinkage or collapse.

Given these observations, as well as the fact that chloroform is toxic to humans, and furthermore that it dissolves many plastics, certain systems and methods of the invention advantageously eliminate the use of chloroform and replace it with additional methanol volume. In certain methods such a dehydrating/staining formulation may comprise 86% Methanol, 9.6% acetic acid, 3.8% of 30mM DAPI in DMSO, 0.0038 g/L eosin. In other methods for dehydration/staining, the formulation comprises 86% methanol, 9.6% acetic acid, 3.8% of 30mM DAPI in DMSO, and 5.5 uM Alexa 594-NETS ester or structural equivalent. Variations on the order of 20% in each of these components are expected to achieve similar results.

The use of acetic acid offers multiple benefits including counteracting tissue shrinkage, improving permeability of cells to reagents, and enhancing fluorescent dye performance in certain dye combinations. In various embodiments, other acids can offer similar benefits. For example, lower concentrations of more powerful hydrochloric acid results in similar effects. However, methods of the invention recognize that increasing the hydrogen ionization potential (lowering the pKa) beyond a certain value places the tissue at risk of degradation in a time-dependent manner. Similarly, it can degrade the fluorescence of eosin, thereby reducing the ability to produce a useful image with fluorescence-based imaging. Accordingly, pKa level is an important consideration for achieving the improved penetration and brighter nuclear signal in combined dehydration/staining step, balanced against the risk of tissue damage from very acidic conditions. In certain embodiments, a pKa range (at room temperature) from approximately 4.82 to 5.2, and more narrowly 4.86 to 5, is desirable in use with the dehydrating/staining solutions and methods described herein. The use of 5-15% acetic acid in methanol, and preferably 10% acetic acid in methanol, can achieve the desired coloration and depth penetration for the disclosed dye combinations in thick tissue specimens without causing undue damage, especially after controlled fixation steps as described above. Additionally, fixation in formalin may increase permeability of cells and may reduce the need for the permeability effect of low pKa. However, low pKa may still be desirable to create a good balance between nuclear and protein dyeing used to mimic the recognizable H&E staining using the imaging methods herein and to maximize the information content of microscopic examination.

### Dye Concentration

For both nuclear and protein dyes, ideal concentrations may depend on a range of factors including: tissue type and the corresponding density of nucleic acids/proteins, formalin fixation time, absorption of one dye emission versus another (e.g., eosin reduces DAPI signal), excitation wavelength (a single excitation wavelength saves time and instrument complexity/cost), excitation power, fluorescent emission separation (how much signal of one dye is collected relative the other for a given excitation power), time of signal integration (how much time is spent at one spot collecting data to improve signal to noise), detector performance characteristics such sensitivity and dynamic range, and the degree of binding and signal augmentation/diminution caused by the staining formula. Based on those considerations, a dehydrating/staining solution of the following composition has been found to provide improved processing and imaging using the methods described herein.

DAPI is a preferred nuclear dye. However, the concentration of 10 micromolar used in earlier applications is too low a concentration for optimal imaging at speed. The signal-to-noise ratio has been found to degrade rapidly as the total amount of dye excited at each position corresponding to an image pixel goes down, which occurs as a result of faster imaging. Thus, with a fast microscope, much higher DAPI concentrations may be used relative to those disclosed in earlier applications discussed herein. In certain embodiments, a concentration of about 1.1 mM DAPI (about 110× higher than disclosed in the earlier-referenced applications and methods discussed below) has been found to produce images of nuclei with no or barely perceptible image degradation at high speed imaging in specimens fixed for less than a few hours, specific to two-photon excitation wavelengths of between about 740 nm and 850 nm, and preferably about 760 nm to about 800 nm, and more preferably 780 nm. DAPI concentration should be minimized while still achieving the desired imaging performance in order to reduce processing costs, as DAPI is a driver of overall cost in the solutions described herein. Accordingly, concentrations of around 550 micromolar may be used to produce acceptable images, but there may be perceptible degradation at high speed. About 800 micromolar is a desired concentration in certain embodiments. In other embodiments, 1.1 mM DAPI is the optimal concentration. In various embodiments, concentration up to about 2.2 millimolar may be used beyond which point test cost may become prohibitive and the binding limits of DAPI are approached. Dye concentration may be varied in certain embodiments based on the tissue sample size. Since the amount of dye used goes up proportionally to sample size, the lowest possible concentration may be preferable in larger samples. Such larger samples also may not need more than one or two optical images, so the reduction in signal that occurs with depth may be less of an issue. Similarly, for rapid imaging of surface such as desired for intraoperative assessment of tissue (i.e. frozen section) only shallow imaging may be needed. For shallow imaging, a lower concentration of 500 μM is sufficient. By contrast, for small samples like biopsies, especially core biopsies, it is desirable to image through 1 mm or more of tissue to get as much visual information as possible. As such, these smaller specimens benefit most from having DAPI concentration between 800 and 1200 micromolar or preferably about 1000-1200 micromolar. There are various formulations of DAPI, including DAPI dilactate and DAPI dihydrochloride, which are essentially equivalent in terms of performance for this purpose. However, in certain embodiments, DAPI dihydrochloride may require a slightly higher concentration relative to other formulations for equivalent effect in this approach. Similarly. Hoechst dyes in its various formulations can substitute for DAPI in certain embodiments with equivalent effect, requiring comparable concentrations to DAPI dihydrochloride or DAPI dilactate.

In certain embodiments, high eosin concentrations may be a problem for protein dense tissue where the eosin binds protein in regions between the objective and the imaging plane. Without being tied to a specific theory, this may occur by eosin absorbing the fluorescence emission of the DAPI, thereby reducing the signal at depth. These effects can be diminished by balancing between the DAPI/Hoechst concentration/signal and the eosin concentration to ensure both channels come out clearly at a specific excitation wavelength and power used. Additionally, relative to prior methods described herein, when using an excitation wavelength of 780 nm (achievable with less expensive lasers), lower concentrations of eosin may be required as eosin excitation is near its peak at that wavelength. As noted above, the dye concentrations (nuclear and protein) should be balanced and one concentration will affect the other. Accordingly, when DAPI concentration is about 1000 μM (as optimized above), a complimentary eosin concentration may be about 5.5 uM. In preferred embodiments, eosin concentration may be at least 3 uM.

In various embodiments, eosin concentration may be more flexible than the DAPI concentration and may be varied by up to an order of magnitude in either direction (0.3 uM-60 uM) while still being usable. Higher concentrations might decrease depth detection of nuclear staining but may be useful for quick staining and near-surface imaging. Alternatively, lower concentrations can still yield sufficient detectable image with high-sensitivity detectors that can be amplified.

### Mucin Detection

Detection of acidic polysaccharides, such as those that comprise the major part of mucins (mucous glycosylated proteins), is useful for histologic evaluation of tissues. Examples include detection of “goblet cells” in the assessment of esophageal reflux disease, characterization of the health of cells in the small and large intestine, and identification of the subtype in solid tumors from a variety of organs.

A surprising advantage of the dehydrating/staining solutions described herein is their ability to strongly stain mucin in a manner that is comparable to the standard special stain employed for such purposes known as Alcian Blue. In certain methods described herein DAPI may be analogous in its binding to hematoxylin, the standard nuclear stain used in physical slides. But hematoxylin is not fluorescent. DAPI binds nucleic acids in a similar manner. Hematoxylin can also bind mucin but it usually does not stain in detectable levels under recommended staining pH and dye concentration conditions, unless specific chemical types and pH conditions are used (e.g. Henwood, Anthony F. “Hematoxylin and eosin staining of mucins of the gastrointestinal tract.” Journal of Histotechnology 40.1 (2017): 21-24.).

Alcian blue is also non-fluorescent, so it cannot be used with available forms of laser-based microscopy. Special stains require additional work and special reagents, and the quality is technique dependent. They also consume tissue which might otherwise be used for other ancillary studies.

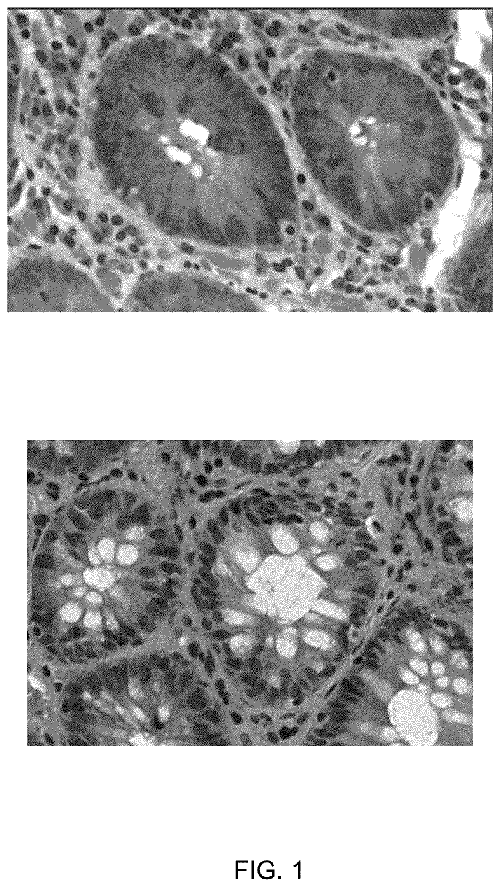

In contrast, some of the formulations described herein can produce a unique visualization of tissues containing acid polysaccharides because they stain strongly with the nuclear dye DAPI when reacted under acidic conditions in alcohol such as the solution formulations described above, where the pKa of the solution is about 4.88. With the image processing techniques described herein, converting two channel fluorescence to H&E-like coloration, the result is a stain type that visually appears similar to a combination of H&E and Alcian Blue. While this combination is not typically performed with physical slides, it offers an opportunity to perform a more complete assessment of tissue, including both standard morphologic assessment and ready quantitation of mucin, with fewer staining steps, while not adversely affecting standard H&E assessment. FIG. 1 shows a comparison of standard H&E stain and the fluorescent nuclear stain methods described herein. The mucin in the pictured epithelial cells is visible using methods of the invention (top) and not noticeably stained using conventional methods (bottom).

### Imaging Analysis

While performing digital imaging of microscopic tissue, speed is an important feature and, therefore, imaging dead space and other inefficiencies can be detrimental to quality imaging. However, missing tissue while scanning could result in mistaken diagnoses so it is important to not sacrifice relevant detail while trying to avoid dead space. Detecting tissue in whole slide imaging typically relies on taking a low power image, then using image analysis techniques to isolate tissue area, then drawing a bounding box around the area.

Techniques described herein recognize that tissues stained with eosin and other general protein-binding dyes fluoresce under excitation from specific wavelengths, that relevant portions of tissue may stain strongly with eosin, and that fluorescent signal can easily be distinguished from non-fluorescent signal. The difference between images taken of stained tissue without excitation and then with excitation indicates the fluorescence containing portion with high signal to noise ratio, which can help separate dirt and other potential noise. Fluorescence imaging alone produces very specific wavelengths which can be isolated and produce sufficient signal to noise for accurate and specific tissue detection.

Taking advantage of these findings, methods of the invention may include taking an image of stained tissue with a standard camera underlight with specific wavelength ranges and, optionally, with a second range of light wavelengths such as red light, or green light, or white light. For example, when an orange/red fluorescing protein dye is used, such as Alexa 594 or Atto 594, then yellow/orange light from, say, a light emitting diode that includes wavelengths in the range of 590 nm (e.g. yellow, “sodium” yellow, amber, or orange LED) can be used to excite the protein dye throughout the entire specimen and a camera can detect the red light emission around 617 nm (orange/red) to identify with high contrast where tissue resides. The desired excitation may also be achieved by other means known in the art, such as using filters that restrict emission wavelengths. Similarly, the desired detection wavelengths can be restricted in the same manner. When eosin is used as a protein dye, then ultraviolet, blue or, green light (range from 440-560 nm, or preferably restricted around 527 nm such as 520-530 nm), can be used to illuminate the tissue, and light emission is collected between 510-640 nm, and optimally in a range that is redder than the emission (such as 530-600 if green LED is used), to improve the isolation of relevant signal by wavelength. In some versions, images can be taken with a green LED excitation. In other versions, images are taken with an amber LED excitation. Alternatively, the image for analysis can be produced by subtracting a 1st image taken under one condition of excitation and detection wavelengths from a 2nd image taken under different excitation and detection wavelengths. For example, an image collected under green LED excitation produces a standard red/green/blue (RGB) image, and the desired signal can be isolated by subtracting one channel from another, such as the blue channel from the red channel, improving the signal to background ratio and facilitating the determination of the region of interest. Methods can also include creating a thresholded mask from this processed image. In some embodiments, the region of interest is determined using a range of hue, saturation, and value from the collected images that corresponds to the dye emission. Optionally, for any of these approaches, a median filter can be employed to reduce the effect of noise on the accuracy of the tissue region. It may also useful to set a minimum connected area to reduce the likelihood that small dirt particles or noise be recognized as tissue, unnecessarily expanding the imaging area. A bounding box can then be created around the fluorescing region of the tissue and the information can be provided to the imaging microscope system to restrict image collection to the relevant areas. The low power imaging data (e.g., standard camera imaging) and bounding box information can be held in the metadata of collected microscopic images.

In various embodiments, an electronic identifier for microscopic specimens may be created based on images collected during processing and pre-imaging or microscopic imaging itself Image analysis can be used to identify unique features such as overall shape or size or the shape or size of sub-regions or features within the tissue sample. Such features can form a digital fingerprint that is associated with the sample, a patient name or laboratory ID number and applied to images thereof. In certain embodiments, the raw image files may be associated with a patient name or ID number and saved in a database such that tissue samples can subsequently be compared to an image or series of images to match features or compared to the aforementioned digital fingerprint of pre-selected features. Such analysis takes advantage of the systems and methods described herein that maintain a sample in a single orientation and configuration for processing and imaging steps, allowing subsequent identification by these methods. Notably, this identification technique is applicable to any image created during processing, pre-imaging, or microscopic imaging, whether it uses fluorescent-, epi-, or trans-illumination using any light wavelengths and image detection method. In other words, it is not restricted to images obtained using the blue or UV light approach described in this application.

In a preferred embodiment, a non-microscopic image of a substantially immobilized biological sample may serve as a unique identifier for cross-reference with a database of sample images. In some embodiments, the biological sample may experience rigid transformations, such as translation or rotation within the field of view. The images may be obtained by a regular camera, such as a CCD camera or CMOS camera. The images may also comprise fluorescence images. The database of sample images may include images acquired by modality, or by another modality, such as by a regular camera or fluorescence image, that is sufficient to enable matching of sample images to those present in the database. The database serves to link such images to other forms of identification. The other forms of identification may comprise a numerical identifier, an optical bar code, a patient name, the name of personnel involved in the collection, processing or evaluation of the tissue sample and images, or any other such information that comprises an alternative mode of sample identification.

The images used to reference the database or for storage in the database may be pre-processed to adjust image parameters such as contrast, brightness, or color balance. Nonlinear processing of the image, such as brightness thresholding, may also be used. A portion of the image data may be used, such as a single color channel, or a single region of the image.

Matching of the input images to images contained in the database may be achieved through any method known to one of ordinary skill in the art, and may comprise image cross-correlation, nearest-neighbor mapping of anchor points or principle components, or machine learning algorithms.

In some embodiments, the biological sample is an intact specimen. In alternative embodiments, the sample may be a slice of biological specimen mounted on a slide. In some embodiments the sample may be embedded in a paraffin block.

Alternatively, a microscopic image of a substantially immobilized biological sample may serve as a unique identifier for cross-reference with a database of sample images. In some embodiments, the biological sample may experience rigid transformations, such as translation or rotation within the field of view. The images may be obtained by a microscope or other device capable of high-magnification imaging of the sample, including a light microscope, a confocal microscope, and multiphoton microscope, a Raman microscope, a stimulated Raman scattering microscope, a coherent anti-Stokes scattering Raman microscope, a side-plane illumination microscope, a total internal reflection microscope, a phase-contrast microscope or other phase-sensitive microscope. The image may be based on transmitted light, reflected light, scattered light or dark-field illumination, fluorescence, second- or third-harmonic light scattering, Raman light scattering, stimulated Raman scattering, coherent anti-stokes Raman scattering.

The microscopic image may be taken at a specified depth within the sample, such as by fluorescence multiphoton microscopy.

The microscopic images used to reference the database or for storage in the database may be pre-processed to adjust image parameters such as contrast, brightness, or color balance. Nonlinear processing of the image, such as brightness thresholding, may also be used. A portion of the image data may be used, such as a single color channel, or a single region of the image. The microscopic image may be reduced in size prior to matching, for example as a thumbnail image.

The database of sample microscopic images may include images acquired by the same modality, or by an alternative modality sufficient to achieve image matching by an algorithm known to one of ordinary skill in the art. Matching of the input images to images contained in the database may be achieved through any method known to one of ordinary skill in the art, and may comprise image cross-correlation, nearest-neighbor mapping of anchor points or principle component analyses, or machine learning algorithms.

In some embodiments, the biological sample may be an intact specimen. In alternative embodiments, the sample may be a slice of biological specimen mounted on a slide.

### Combined Collagen and Nuclear/Protein Stain Detection

Despite the time-tested benefit in prediction and characterization of disease states based on collagen assessment with trichrome staining, major limitations are associated with this common procedure. One is poor reproducibility of visual estimation and high inter- and intra-observer variability. Attempts at quantitative image-based morphometric analysis based on imaging of trichrome, use of immunohistochemistry, and other collagen stains, such as Sirius Red, have demonstrated improved precision and correlation with other function measures, but require additional time and labor for use. This additional effort compounds the already highly laborious and time-consuming preparation of physical thin sections of tissue. They are also only routinely available for single slice evaluation, which statistically would be expected to limit accuracy and thereby overall value to clinical management. As such, there exists a need for more comprehensive, precise, and quantifiable assessment of collagen content in tissues, ideally in a manner that reduces overall effort.

Advances in confocal microscopy, multiphoton microscopy, and other optical sectioning techniques have expanded the ability to perform three dimensional imaging in tissue specimens, generating a 3D perspective without the embedding, sectioning, scanning, and image processing work associated with traditional serial sectioning, albeit to a limited depth. Over the past decade, optical sectioning methods have been combined with chemical processing to reduce light scatter in tissue, markedly improving deep tissue imaging and extending the depth of tissue reconstructions from microns to millimeters using many of the techniques discussed herein.

Light scattering in tissue is due to refractive index differences between membranes, proteins, and water, as a result of which light is redirected, reducing the ability to pinpoint the source. Clearing techniques for reducing light scattering fall into two general categories, removal of lipid membranes and replacement of water with high refractive index fluids. The latter has the advantage of preserving stainable structures as well as requiring far less time and work. Among clearing solutions, the inexpensive and safe compound mixture of benzyl alcohol/benzyl benzoate in a 1:2 ratio (BABB), a modification by Murray of a clearing compound originally developed more than a century ago, matches closely the refractive index of dehydrated proteins and membranes, approximately Nd 1.55. Ethyl cinnamate has similar properties and can be employed for the same purpose.

An additional property of multiphoton laser excitation called second harmonic generation (SHG) has been used to selectively visualize the collagen content in tissues as described in U.S. patent application Ser. No. 17/000,994. The pulsed laser takes advantage of a property specific to non-centrosymmetric molecules. In tissue samples such molecules are found primarily in collagen. The SHG process involves the use of a pulsed laser with pulses short enough that the instantaneous intensity is sufficient for two photons to simultaneously stimulate a collagen molecule. This occurs with high probability only in a small volume dictated by the focusing capability of an objective lens, achieving high spatial selectivity in three-dimensional space. When this happens, two photons are combined and emitted as a single photon of twice the energy (half the wavelength). According to the invention, this specific, non-fluorescent phenomenon can be combined with clearing to augment the quantity of accessible collagen data, and thereby the precision of overall quantitation, while also enabling three-dimensional characterization of collagen architecture within a sample.

There are specific challenges that impose difficulties in routinely implementing SHG detection simultaneously with H&E-like imaging (e.g., using fluorescent dyes as described herein) in thick specimens, however. Certain tissues such as the liver have a very high protein content which is related to its high metabolic activity as a primary detoxifying organ of the body. Ideally, the SHG collagen visualization would be accomplished concurrently with protein and nucleic acid imaging, reducing overall work and processing time while maximizing informational content derived with imaging. However, when exciting this collagen signal with pulsed lasers using wavelengths between about 750 nm and 900 nm, the SHG signal can be severely attenuated at depth if the concentration of eosin, staining the protein, is high. This is somewhat surprising because eosin is not expected to absorb light in either these excitation wavelengths nor in the resulting SHG signal wavelengths (exactly half the excitation wavelength). But our observations have verified that high protein staining with eosin does produce an attenuation of signal of SHG, for which absorption is a possible explanation. It is still helpful to visualize the anatomic structures surrounding the SHG signal so as to contextualize whether the signal is from normal fibrous tissue or a pathologic reaction. Intrinsic fluorescence may be used to accomplish this goal, but some protein fluorescent dye facilitates this detection. Accordingly, in certain embodiments, eosin concentration may be minimized during staining to the lowest level that still allows visualization of microscopic protein-rich structures in order to increase the depth at which collagen signal can be simultaneously detected. For liver specimens, using staining methods, processing reagents, and multiphoton imaging systems as described herein, using eosin concentrations of around 0.02% of stock solutions during dyeing steps yields a balance of protein signal for adequate reproduction of H&E stains while permitting imaging of collagen SHG at several hundred micrometers.

A physical property of SHG is that the intensity of the signal is often significantly higher in the transmitted direction (parallel to the direction of excitation light). If one attempts to collect an SHG signal propagating in the reverse direction (epi-detection), for example using the same objective lens for excitation and detection, the SHG signal is typically attenuated. Unfortunately, the use of this geometry is often a function of efforts designed to minimize the cost and complexity of microscopy systems and, in some cases, may be dictated by constraints in the design of the device holding the sample as well as the mechanical elements in a functional multiphoton microscope. In such circumstances, any fluorescent dye with the potential to emit signal within the specific wavelength of detection of the SHG signal can obscure the signal of interest, reducing the accuracy of SHG imaging. For example, when using DAPI or Hoechst for nuclear staining, the broad bright fluorescence of these dyes in short visible wavelengths can easily overwhelm the SHG signal, even when using very narrow range emission filters, meant to minimize the contribution from those dyes. Additionally, eosin has a proportionally more relevant detrimental effect on the excitation signal when a weak SHG signal is being detected at depth in the epi-detection mode, and eosin may also absorb emitted SHG signal, creating in effect an absorptive filter of the relevant signal.

To avoid these issues, certain methods of the invention may include approaches for processing tissue specimens in preparation for imaging with a multiphoton microscope under conditions of matched refractive index for imaging at depth with high resolution and contrast, providing optical section information on collagen content and distribution, followed by processing tissue with fluorescent dyes and imaging in a manner that can accurately reproduce the type of information obtained from thin tissue sections. Using methods described herein, collagen information may be initially retrieved before the same specimen, optionally in the same container, is subsequently stained and imaged using an optical sectioning microscope, which optionally may be the same multiphoton microscope used to obtain the collagen information.

Given the limitations noted above for standard approaches to collagen imaging and incorporation of SHG signal detection in a multiphoton microscope, in certain embodiments, a sample may be processed to clearing without the use of any dyes. This process can occur by any one of the methods known for chemical clearing, meaning replacing the water content with a fluid with a high refractive index (including the methods described herein). This refractive index produces sufficient clarity to usefully detect SHG at tens to hundreds of microns of depth when the fluid refractive index is about 1.47 or higher. Ideally the refractive index of the fluid is 1.5 or higher. Most ideally the refractive index of the fluid is 1.545, or about 1.55, when measured at 780 nm. In a preferred embodiment the process involves optionally fixing the tissue in formalin, then placing the sample in methanol, then placing the sample in BABB before subjecting to SHG analysis for collagen characterization.

The sample can then be exposed to alcohol, or other tissue processing compounds known to those with ordinary skill in the art such as xylene which are miscible with both organic solvents and alcohols, to remove the clearing compound, optionally in the same container in which the clearing and imaging had been performed. Exemplary containers for tissue sample processing are described in U.S. Pat. No. 10,527,528, the content of which is incorporated herein by reference. The sample can then be treated with fluorescent dyes in an alcoholic solution (e.g., using any of the dyes and/or dehydrating/staining solutions and methods described herein) and cleared, processed, and subjected to the optical sectioning fluorescent microscopy methods described herein. Accordingly, clear SHG analysis of collagen content at depth can be obtained in addition to the H&E-type fluorescent staining analysis described herein from the same tissue sample, providing a more complete profile for diagnostic and prognostic purposes.

As discussed above, various systems and methods described herein relate to improvements and modifications to earlier described image analysis methods used to produce images essentially indistinguishable from traditional histology stains with fluorescent dyes and optical sectioning microscopy. Aspects of those methods are described below and provide images of samples that mimic common pathology stains, resulting in the accurate and efficient interpretation of the images. Contrary to standard color separation algorithms, these methods invert the color separation techniques using the fluorescence of the sample, whether inherent or resulting from a fluorescent dye, to faithfully recreate images comprising the expected colorization of tissues resulting from common stains such as hematoxylin/eosin and wright/giemsa, allowing the images to be easily interpreted by pathologists. Contrary to past efforts of pseudo-colorization, the methods described herein use exponential conversion equations, more closely matching the optical qualities of fluorescence emission to those of light absorption with traditional illumination of thin sections. As demonstrated herein, the methods of the present invention result in the production of images that have resolution and fields of view similar to those produced using current histological methods, provide a contrast similar to that obtained with commonly used histologic stains, and permit subsequent traditional processing without apparent adverse effects. The multichannel method described herein provides straightforward pseudocolorization that represents morphology in an analogous method to traditional stains, allowing pathologists to easily recognize salient histologic features.

With biopsies, there is often a trade-off between keeping sufficient tissue for additional stains or molecular analysis and adequate hematoxylin and eosin (H&E) histology. The necessarily sparse sampling of traditional physical wax-embedding and cutting histology techniques can miss important features. For example, colonic polyps may be missed, small foci of prostate cancer may be non-diagnostic, and focal renal lesions may be unapparent. This problem is compounded by the need to discard initial block shavings for complete sections, particular in imperfectly embedded specimens. The methods described herein obviate these issues that occur when using current histological methods while permitting image reconstruction of entire or deep portions of biopsy specimens.

In the case of rapid analysis, the many artifacts and difficulties associated with tissue freezing and sectioning can be overcome by visualizing un-frozen, uncut tissue with the process presented. In contrast to other related efforts to visualize un-frozen, un-cut fresh tissue with optical sectioning microscopes, the process described here fully addresses challenges that stem from poor refractive index matching and slow dye penetration that preclude imaging of adequate resolution and depth for practical diagnosis with any other known technique.

### Methods

In one aspect, the present invention provides methods of processing and imaging a histological sample. In one embodiment, the method comprises the step of obtaining a sample. The sample can generally be any type of sample. For example, the sample can be a cell or group of cells, an organism, a tissue, cell lysates, a cell culture medium, a bioreactor sample, and so on. In a preferred example, the sample is a tissue sample. In another embodiment, the sample is a fluid sample in which the cellular component has been concentrated such as by centrifugation or filtering. Non-limiting examples of tissues include skin, muscle, small bowel, large bowel, breast, esophagus, heart, kidney, lung, liver, skin, placenta, prostate, pancreas, uterus, bone, bone marrow, brain, stomach, muscle, cartilage, lymph node, adipose tissue, tonsil, gall bladder, and spleen, as well as tumor tissue of any cell type and the cellular component of cerebrospinal fluid, pleural fluid, ascites fluid, or synovial fluid. In one embodiment, the tissue is liver tissue. In another embodiment, the tissue is kidney tissue. In another embodiment, the tissue is breast tissue. In another embodiment, the tissue is prostate tissue. The sample may be obtained through any method known in the art, as would be understood by one skilled in the art. In some embodiments, the sample is obtained during surgery, biopsy, fine needle aspiration, culture, or autopsy. In one embodiment, the sample is a fresh sample. In another embodiment, the sample is a fixed sample. In one embodiment, the tissue sample is fixed prior to obtaining the tissue sample.

Methods of the invention may comprise obtaining a sample and contacting the sample with a fixation solution. In a preferred embodiment, the sample is a tissue sample. In certain embodiments, the fixation solution comprises at least one dehydrant and at least one fluorescent dye. In some embodiments, methods may comprise clearing the sample by contacting the sample with a clearing solution to provide increased depth and clarity for imaging the sample. In some embodiments, the sample is fixed prior to being contacted with a solution comprising a fixative or dehydrant and at least one fluorescent dye. In another embodiment the fresh tissue is placed directly in a combination fixation/dehydration fluid with dye or dyes. In some embodiments, the step of imaging the sample is performed in combination with an additional imaging method, such as second harmonic generation (SHG). In certain embodiments, specimens can be partially fixed for intra-operative evaluation or for any other clinical scenario where a fast visual examination is desired. Partially fixed specimens may be fully fixed at a later time. In another embodiment, specimens can be partially cleared for intra-operative evaluation or for any other clinical scenario where a fast visual examination is desired. Partially cleared specimens may be fully cleared at a later time.

In one aspect, the method of the present invention further comprises the step of dehydrating the sample. Dehydration facilitates the removal of water from a sample so that clearing agents with low water solubility can subsequently be used. It should be appreciated that a dehydrant or a dehydration solution may also be used as a fixative or for fixing a sample. As used herein, the term “dehydrant” refers to a water-miscible anhydrous fluid. Non-limiting examples of dehydrants include alcohols such as methanol, ethanol, and propanol. In one embodiment, the dehydrant is methacarn. In another embodiment, the dehydrant is methanol. In one embodiment, the dehydration step functions as a fixative and takes place without prior fixation of the sample. In other embodiments, the dehydration step is performed after fixation of the sample. In one embodiment, the dehydration step is performed after fixation of the sample using a fixation solution comprising formalin.

The dehydration step can be performed for any suitable length of time. The length of time can generally be any length of time suitable for rendering the sample, or a portion of the sample, miscible with the clearing agent. The length of time can also generally be any length of time suitable for preserving the sample or preserving a portion of the sample. In certain embodiments the period of time may be from about 1 minute, about 5 minutes, about 10 minutes, about 15 minutes, about 30 minutes, about 1 hour, about 90 minutes, about 2 hours, about 3 hours, about 4 hours, about 5 hours, about 6 hours, about 12 hours, or about 24 hours. In one embodiment, the dehydration step is performed over a period of time about 1 hour. In another embodiment the dehydration step is performed over about 12 to 16 hours.

In one aspect, the method of the present invention comprises the step of fixing the sample. The tissue sample may be fixed using any method known in the art, as would be understood by one skilled in the art. In one embodiment, the sample is fixed by contacting the sample with a fixative. In another embodiment, the sample is fixed by contacting the sample with a fixation solution. In one embodiment, the fixation solution comprises at least one fixative. In one embodiment, the fixative is a dehydrant. In another embodiment, the fixation solution is a dehydrant. In another embodiment, the fixation solution comprises at least one fixative and at least one permeant. Non-limiting examples of fixatives include aldehydes (e.g., formaldehyde (paraformaldehyde, formalin), glutaraldehyde, acrolein (acrylic aldehyde), glyoxal (ethanedial, diformyl), malonaldehyde (malonic dialdehyde), diacetyl (2,3-butanedione), and polyaldehydes; alcohols (i.e., protein-denaturing agents; e.g., acetic acid, methanol, ethanol), polyvinyl alcohols, heavy metal oxidizing agents (i.e., metallic ions and complexes; e.g., osmium tetroxide, chromic acid); agents of unknown mechanism, such as chloro-s-triazides, cyanuric chloride, carbodiimides, diisocyanates, diimido esters, diethylpyrocarbonate (diethyl oxydiformate, ethoxyformic anhydrate), picric acid, mercuric chloride (corrosive sublimate, bichloride of mercury), and other salts of mercury, and acetone. In one embodiment, a combination of fixatives is used. Such combinations give rise to commonly termed formulations known to those in the art, such as Carnoy's fixatives, methacarn, Wolman's solution, Rossman's fluid, Gendre's fluid, Bouin's fluid, Zenker's fluid, Helly's fluid, B5 fixative, Susa fluid, Elftman's fixative, Swank and Davenport's fixative, Lillie's alcoholic lead nitrate, and cetylpyridinium chloride (C.P.C.). Additives can include, but are not limited to, such entities as tannic acid, phenol, transition metal salts (zinc), lanthanum, lithium, potassium. In one embodiment, the fixative is methacarn. In another embodiment, the fixative is formalin. In another embodiment, the fixative is an alcohol. In another embodiment, the fixative is methanol. In another embodiment, the fixative is a polyvinyl alcohol. In another embodiment, the fixative is formaldehyde. In another embodiment, fixation of the sample occurs ex vivo.

In some embodiments, at least one fluorescent dye is added to the sample during the fixation step, resulting in simultaneous fixing and staining of the sample. In other embodiments, at least one fluorescent dye is added to the sample during the dehydration step, resulting in simultaneous dehydration and staining of the sample. The incorporation of a fluorescent dye obviates the need for post-processing staining, which is a time-consuming step of traditional sample preparation. The fluorescent dye may be added directly to the sample during the fixation step. For example, the fluorescent dye may be added to the fixation solution. In another embodiment, the fluorescent dye is added to the sample after completion of the fixation step. In one embodiment, the fixation solution comprises a fixative and a fluorescent dye. In another embodiment, the fixative solution comprises at least one dehydrant and at least one fluorescent dye. In one embodiment, the fluorescent dye is added directly to the sample during the dehydration step. In one embodiment, the method of processing a tissue sample comprises the steps of obtaining a tissue sample, and contacting the tissue sample with a fixative solution comprising at least one dehydrant and at least one fluorescent dye.

The skilled artisan will understand that the present invention contemplates the use of any fluorescent dye that is compatible with the fixation step. Examples of fluorescent dyes include, but are not limited to, the nucleic acid stains sold under the trademarks POPO-1, TOTO-3, SYTOX, and SYTO by ThermoFisher Scientific (Waltham, Massachusetts), TAMRA, BOXTO, BEBO, SYBR DX, dyes sold under the name Alexa Fluor dyes (e.g. Alexa Fluor 594, Alexa Fluor 594-NHS ester, Alexa Fluor 633, Alexa Fluor 633-NHS ester, Alexa Fluor 568, Alexa Fluor 568-NHS ester, Alexa Fluor 610, Alexa Fluor 610-NHS ester) as well as chemical equivalents sold under names AZDye, XFD, Aurora Fluor and others, fluorescein, rhodamine, rhodamine derivative dyes sold under the name ATTO (ATTO-TEC Siegen, Germany) (e.g. ATTO 590, ATTO 594, ATTO 610, ATTO 633, etc), propidium idodide, Hoechst dyes, tetramethylrhodamine, R-phycoerythrin, Cy-3, Cy-5, Cy-7, sulforhodamine 101 acid chloride, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 8-Anilino-1-naphthalenesulfonic acid ammonium salt (ANS), 4-acetamido-4′-isothiocyanatostilbene-2,2′ disulfonic acid, acridine, acridine isothiocyanate, acridine orange (N,N,N′;N′-tetramethylacridine-3,6-diamine), R-amino-N-(3-vinylsulfonyl)phenylnapthalimide-3,5, disulfonate (Lucifer Yellow VS), N-(4-anilino-1-naphthyl)maleiniide, anthranilamide, Brilliant Yellow, coumarin, 7-ammo-4-methylcoumarm, 7-amino-4-trifluoromethylcouluarin (Coumarin 151), cyanosine, 2-(4-amidinophenyl)-1H-indole-6-carboxamidine (DAPI), 5′,5′-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin- 2-yl) aminofluorescein (DTAF), QFITC (XRITC), fluorescamine, 1R144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, thiazole orange, riboflavin, rosolic acid, and terbium chelate derivatives. In one embodiment, the fluorescent dye is eosin. In another embodiment, the fluorescent dye is DAPI. In another embodiment, the fluorescent dye is the nucleic acid stain sold under the trademark SYTOX green by ThermoFisher Scientific (Waltham, Massachusetts). In another embodiment, the fluorescent dye is acridine orange. In another embodiment, the fluorescent dye is rhodamine B. In another embodiment, the fluorescent dye is a nucleic acid stain sold under the trademark SYTO by ThermoFisher Scientific (Waltham, Massachusetts). In another embodiment, the fluorescent dye is propidium iodide. In another embodiment, the fluorescent dye is a Hoechst dye.

In certain embodiments, the fluorescent dye can selectively stain a particular organelle or component of a cell. In one embodiment, the fluorescent dye is a nuclear dye. Non-limiting examples of nuclear dyes include DAPI, nucleic acid stains sold under the trademark SYTOX by ThermoFisher Scientific (Waltham, Massachusetts), nucleic acid stains sold under the trademark SYTO by ThermoFisher Scientific (Waltham, Massachusetts), propidium iodide, acridine orange, and Hoechst dyes. In one embodiment, the nuclear dye is DAPI. In another embodiment, the fluorescent dye is a protein dye. Examples of protein dyes include, but are not limited to, eosin, Rhodamine B (RhB), and ANS. In some embodiments, the fluorescent dye is an NHS ester of a rhodamine derivative, used as a general protein dye. In some embodiments the fluorescent dye is chemically equivalent to the compound sold as Alexa Fluor 594-NHS ester. In other embodiments the protein dye is ATTO 594-NHS ester or its chemical equivalent. In some embodiments the protein fluorescent dye is Alexa Fluor 610-NHS ester or its generic equivalent. In another embodiment, the fluorescent dye is a chemical equivalent of the compound sold under the name Alexa Fluor 633 NHS ester. In another embodiment, intrinsic fluorescence of the cell is used to image the cellular protein. In another embodiment, the fluorescence is generated from a combination of at least one nuclear dye and at least one protein dye. In another embodiment, the fluorescence is generated from a nuclear dye and intrinsic fluorescence. In one embodiment, the at least one protein dye is eosin. In another embodiment, the at least one protein dye is Alexa Fluor 594-NHS ester or its chemical equivalent.

In certain embodiments, a morphology preservative is added to the sample during the fixation step and/or the dehydration step. The morphology preservative enhances maintenance of the nuclear structure of the cells, in that it maintains cell membranes intact for subsequent cytological staining and/or reduces shrinking or swelling during fixation or dehydration. Any morphology preservative that is compatible with the fixation step may be used in the invention, as would be understood by one of ordinary skill in the art. Non-limiting examples of morphology preservatives include acetic acid, trichloroacetic acid, formaldehyde, dioxane, chloroform, and the like. In one embodiment, the morphology preservative is chloroform. The morphology preservative may be added directly to the sample during the fixation step. Alternatively, the morphology preservative may be added to the fixation solution. In one embodiment, the fixation solution comprises about 0% to about 50% of a morphology preservative. In another embodiment, the fixation solution comprises about 5% to about 15% of a morphology preservative. In another embodiment, the fixation solution comprises about 10% of a morphology preservative. In another embodiment, the fixation solution comprises about 20% to about 40% of a morphology preservative. In a preferred embodiment, the fixation solution comprises about 30% of a morphology preservative.

In some embodiments, a permeation enhancer is added to the sample during the fixation step and/or the dehydration step. The permeation enhancer accelerates the access of dye to the deeper portion of the sample, while overall improving the dyeing process. The permeation enhancer also accelerates the penetration of fixative, dehydrant, and/or clearing agent. Non-limiting examples of permeation enhancers include acids such as acetic acid, methacarn comprising acetic acid, sulphoxides such as dimethylsulfoxide (DMSO), azone, pyrrolidones, propylene glycol, fatty acids, essential oils, phospholipids, s-collidine, and surfactants such as Tween. In one embodiment, the fixation solution comprises about 0% to about 75% of a permeation enhancer. In another embodiment, the fixation solution comprises about 0% to about 25% of a permeation enhancer. In another embodiment, the fixation solution comprises about 5% to about 15% of a permeation enhancer. In a preferred embodiment, the fixation solution comprises about 10% of a permeation enhancer.

In some embodiments, the permeation enhancer is at least one acid. In some embodiments, the acid is an organic acid. Non-limiting examples of organic acids include acetic acid, glacial acetic acid, lactic acid, propionic acid, butyric acid, succinic acid, citric acid, 3-hydroxypropionic acid, glycolic acid, or formic acid. In one embodiment, the acid is acetic acid. Acetic acid is useful for enhancing the speed of fixation, which is important for rapid sample processing, while also significantly improving the quality and depth of images from cleared samples using any type of sectioning image modalities. Acetic acid is also useful for lysing red blood cells, which allows for removal of heme pigment, which is a significant deterrent to clarity of the sample by virtue of its broad light absorption characteristics in the typical workable wavelength range of routine fluorescent imaging. In one embodiment, the step of fixing the sample further comprises the addition of a lysing agent to the sample. In a particular embodiment, the step of fixing the sample further comprises the addition of a red blood cell lysing agent to the sample. In another embodiment, the step of dehydrating the sample further comprises the addition of a lysing agent to the sample. In another embodiment, the fixative solution further comprises a red blood cell lysing agent. In a preferred embodiment, the red blood cell lysing agent is acetic acid. In other embodiments, the acid is an inorganic acid. The solution may further comprise at least one organic solvent. Non-limiting examples of organic solvents include methanol, absolute methanol, chloroform, dichloromethane, ethanol, isopropanol, acetone, ethyl acetate, acetonitrile, hexane, hexene, octane, pentane, cyclohexane, iso-octane, xylene (ortho, meta, or para), and 1-hexene. In one embodiment, the organic solvent is absolute methanol. Methanol is useful for tissue processing by arresting enzymatic function and degradation while maximally preserving genetic and proteomic information. In another embodiment, the organic solvent is chloroform. In one embodiment, the solution comprises two organic solvents and an acid. As a non-limiting embodiment, the fixative solution comprises about 60% absolute methanol, about 30% chloroform, and about 10% glacial acetic acid, which is also known as methacarn. When in combination with fluorescent dyes and clearing, methacarn may be useful for deep fluorescent tissue section imaging of human samples for histologic evaluation, and for creating the contrast needed for accurate histologic evaluation.

The fixation step may be performed under any condition that promotes rapid tissue processing, such as conditions that increase the rates of chemical reaction and diffusion, as would be understood by one of ordinary skill in the art. In some embodiments, the fixation step is performed at an elevated temperature. As used herein, the term “elevated temperature” refers to temperatures above those experienced in the earth's atmosphere, preferably above 30° C. In one embodiment, the elevated temperature ranges from about 20° C. to about 75° C. In another embodiment, the elevated temperature ranges from about 35° C. to about 50° C. In another embodiment, the elevated temperature is about 45° C. In a non-limiting example, the fixation, dehydration, and/or staining is performed under microwave irradiation for the purpose of accelerating diffusion, chemical reaction, or temperature.

The fixation step can be performed for any suitable length of time. The length of time can generally be any length of time suitable for preserving the sample. In certain embodiments, the period of time may be from about 1 minute, about 5 minutes, about 10 minutes, about 15 minutes, about 30 minutes, about 1 hour, about 90 minutes, about 2 hours, about 3 hours, about 4 hours, about 5 hours, about 6 hours, about 12 hours, or about 24 hours. In one embodiment, the fixation step is performed over a period of time about 1 hour. In another embodiment the fixation step is performed over about 12 to 16 hours. In another embodiment the fixation step is performed over the course of weeks to years.

In one embodiment, the method of the present invention comprises the step of simultaneously fixing, dehydrating, and staining the sample by contacting the sample with a dehydration solution comprising at least one dehydrant and at least one fluorescent dye. In another embodiment, the method of the present invention comprises the step of simultaneously dehydrating and staining the sample by contacting the sample with a dehydration solution comprising at least one dehydrant and at least one fluorescent dye.

In another aspect, the method of the present invention further comprises the step of clearing the sample. Clearing the sample provides increased depth and clarity of imaging of the sample. In one embodiment, the clearing step is performed in absence of a fixation step. In some embodiments, the step comprises clearing the sample by contacting the sample with a clearing solution. As a non-limiting embodiment, the sample is cleared by replacing water with a clearing solution that has a higher refractive index than water that more closely resembles that of proteins and organelles, thereby drastically reducing light scattering and enabling imaging depths of millimeters instead of micrometers. In one embodiment, the clearing solution comprises at least one solvent. Any solvent may be used in the clearing solution, as long as the overall refractive index of the clearing solution is higher than the refractive index of water and the solvent does not damage the morphology of cellular components of the sample. In one embodiment, the refractive index of the clearing solution ranges front about 1.4 to about 1.6. In another embodiment, the refractive index of the clearing solution ranges from about 1.33 to about 1.49. In another embodiment, the refractive index of the clearing solution is greater than about 1.4. In another embodiment, the refractive index of the clearing solution is greater than about 1.5. In one embodiment, the clearing solution further comprises an agent that is water soluble and has a high refractive index, such as a high-concentration sugar solutions.

In some embodiments, the solvent is an organic solvent. Non-limiting examples of organic solvents useful as clearing agents include, benzyl alcohol, benzyl benzoate, xylene, limonene, benzene, toluene, chloroform, petroleum ether, carbon bisulfide, carbon tetrachloride, dioxane, glycerol, dibenzyl ether, clove oil, and cedar oil. In one embodiment, the solvent is benzyl alcohol. In another embodiment, the solvent is benzyl benzoate. In another embodiment, the solvent is xylene. In another embodiment, the solvent is glycerol. In another embodiment, the solvent is a sugar solution. In another embodiment, the solvent is dibenzyl ether. In another embodiment, the solvent is hexane.

In some embodiments, the clearing solution comprises a first solvent and a second solvent. In one embodiment, the ratio of the first solvent to the second solvent ranges from about 100:1 to about 1:100. In another embodiment, the ratio of the first solvent to the second solvent ranges from about 10:1 to about 1:10. In another embodiment, the ratio of the first solvent to the second solvent ranges from about 5:1 to about 1:5. In a preferred embodiment, the ratio of the first solvent to the second solvent is about 1:2. In some embodiments, the solvent is an organic solvent. In a particular embodiment, the clearing solution comprises benzyl alcohol and benzyl benzoate. In one embodiment, the ratio of benzyl alcohol to benzyl benzoate is about 1:2.

The clearing step may be performed under any condition that promotes rapid clearing of the sample, as would be understood by one of ordinary skill in the art. In some embodiments, the clearing step is performed at an elevated temperature. In one embodiment, the elevated temperature ranges from about 20° C. to about 75° C. In another embodiment, the elevated temperature ranges from about 35° C. to about 50° C. In another embodiment, the temperature is about 22° C.

The clearing step can be performed for any suitable length of time. The length of time can generally be any length of time suitable for achieving sufficient reduction in light scattering to enable imaging to the desired depth in the sample. In certain embodiments, the period of time may be from about 1 minute, about 5 minutes, about 10 minutes, about 15 minutes, about 30 minutes, about 1 hour, about 2 hours, about 3 hours, about 4 hours, about 5 hours, about 6 hours, about 12 hours, or about 24 hours. In one embodiment, the clearing step is performed in about 2 minutes to about 1 hour. In one embodiment, the clearing step is performed in about 2 minutes. In one embodiment, the clearing step is performed in about 30 minutes. In one embodiment, the clearing step is performed in about 12 hours.

In one embodiment, the clearing step further comprises the step of adding a solvent to the sample prior to adding the clearing solution. In some embodiments, the solvent is an organic solvent. In one embodiment, the solvent is an alcohol. The alcohol is useful for dehydrating the sample. Non-limiting examples of alcohols include methanol, ethanol, propanol, isopropanol, butanol, isobutanol, ethyl butanol, /-butanol, dioxane, ethylene glycol, acetone, and amyl alcohol. In a preferred embodiment, the solvent is methanol. In one embodiment, the solvent is added in combination with a permeation enhancer. Non-limiting examples of permeation enhancers include acetic acid, polyethylene glycol (PEG), mono- and dimethyleneglycol, propylene glycol, polyvinyl pyrrolidone, or the like, surfactants such as dimethyl sulfoxide (DMSO), polyoxyethylene sorbitan esters (e.g., TWEEN such as TWEEN 80), sodium dimethyl sulfosuccinate, mild household detergents, or the like. In one embodiment, the permeation enhancer is selected from the group consisting of acetic acid, DMSO, and TWEEN. The addition of a solvent in combination with a permeation enhancer increases the rate of clearing with BABB by improving miscibility and permeability, and also eliminates the need for gradual gradient steps of decreasing alcohol concentration.

In part, the present invention provides a method of rapidly processing a tissue sample. The length of time can generally be any length of time suitable for fixing the sample and clearing the sample. The length of time can also generally be any length of time suitable for fixing a portion of the sample and clearing a portion of the sample. In certain embodiments, the period of time may be from about 1 minute, about 5 minutes, about 10 minutes, about 15 minutes, about minutes, about 1 hour, about 2 hours, about 3 hours, about 4 hours, about 5 hours, or about 6 hours. In one embodiment, the steps of fixing the sample and clearing the sample are performed in about 1 hour to about 2 hours. In one embodiment, the steps of fixing the sample and clearing the sample are performed over a period of time about 1.5 hours. In another embodiment, the steps of fixing the sample, dyeing the sample, and clearing the sample are performed over a period of time of about 3 hours.

In another aspect, the method of the present invention further comprises the step of imaging the sample. In one embodiment, the step of imaging the sample further comprises producing a visual image of the tissue sample. The sample may be imaged using any imaging method compatible with the sample processing methods described herein. Preferred imaging methods include fluorescence-based optical sectioning imaging methods. Contrary to destructive 3-D histology approaches such as pigmented plastic embedding systems and whole slide imaging (WSI), these fluorescence-based methods are non-destructive, allowing for the preservation of samples for ancillary studies such as immunostains and molecular studies. Examples of fluorescence-based sectioning imaging methods include, but are not limited to, multiphoton microscopy (MPM), side-plane illumination microscopy, traditional confocal microscopy, spinning disk confocal microscopy, structured illumination microscopy, and the like. In animal tissue (Parra et al., 2010, J. Biomed. Opt. 15:036017-036017-5; Vesuna et al., 2011, J. Biomed. Opt. 16:106009-106009-6; Fu et al., 2009, Gastroenterology 137:453-465), the depth of imaging can be increased over samples prepared using more traditional methods, such as formalin fixing, by combining MPM with optical clearing. In one embodiment, the sample is imaged using multiphoton microscopy (MPM). In another embodiment the sample is imaged using confocal microscopy. In another embodiment, the sample is imaged using structured illumination microscopy. In another embodiment the sample is imaged using selective plane illumination microscopy. In another embodiment the sample is imaged using deconvolution microscopy. In another embodiment the sample is imaged using super-resolution microscopy. In another embodiment, the sample is imaged using side-plane illumination microscopy. In another embodiment, the sample is imaged using spinning disk confocal microscopy. In one embodiment, the method of imaging a tissue sample comprises the steps of obtaining a tissue sample, contacting the tissue sample with a fixative solution comprising at least one fluorescent dye, contacting the tissue sample with a clearing solution, and producing a tissue sample image by measuring intensity values of the fluorescence of the tissue sample, and converting the intensity values to effective optical densities, such that the optical densities recreate the coloration of a stain in a produced image of the tissue sample.

The imaging methods of the present invention provide a method for image analysis that allows reproduction of images essentially indistinguishable from traditional histology stains. In one embodiment, the method involves a multichannel approach, wherein intensity values of fluorescence from the sample are converted to optical densities using an exponential pseudo-coloring process, which is an inversion of a logarithmic pseudo-coloring process, wherein intensity values of fluorescence are converted to optical densities in red, green, and blue channels. In one embodiment, the step of imaging the sample further comprises the steps of measuring intensity values of the fluorescence of the sample and converting the intensity values to effective optical densities recreate the coloration of a stain in the sample image. In one embodiment, the intensity values of one or more fluorescence channels are converted to effective optical densities in one or more pseudo-color display channels using an exponential pseudo-coloring process, wherein the equation that results in optical densities includes a constant to the power of the intensity values from fluorescence, as would be understood by one of ordinary skill in the art. Numerically:

Ch1=C₁\hat{ }(a₁N+b₁P)

Ch2=C₂\hat{ }(a₂N+b₂P)

Ch3=C₃\hat{ }(a₃N+b₃P)

where Ch1, Ch2, Ch3 are color display channels, such as Red, Green and Blue; C₁, C₂, C₃ are positive constants; a₁, a₂, a₃, b₁, b₂, b₃ are constants that may be positive or negative; and N and P are fluorescence intensities recorded from different fluorescence channels. In one embodiment, the intensity values are converted to effective optical densities using an exponential pseudo-coloration process.

For an example of a logarithmic color deconvolution process, see Ruifrok and Johnston, 2001, Anal. Quant. Cytol. Histol. 23:291-299, which is incorporated by reference herein in its entirety.

In one embodiment, the fluorescence is intrinsic fluorescence from the sample. In another embodiment, the fluorescence is fluorescence from the fluorescent dye. In one embodiment, the fluorescent dye is a nuclear dye. In another embodiment, the fluorescent dye is a protein dye. The number of channels used may be varied as needed to achieve the desired image, as would be understood by one skilled in the art. In one embodiment, the number of channels is two channels. In a specific embodiment, the two channels are an intrinsic fluorescence channel and a fluorescent nucleic acid dye channel. In another embodiment, the two channels are a fluorescent nucleic acid dye channel and a fluorescent protein staining channel. In a non-limiting example, the intrinsic fluorescence, emanating primarily from cross-linked proteins and corresponding to the staining typically achieved by protein stains such as eosin, can be augmented by use of formalin as the fixative, a feature that facilitates the reproduction of normal coloration by improving signal to noise of this channel and facilitating separation from nucleic acid fluorescence.

The imaging method of the present invention also provides images of samples that mimic common pathology stains, resulting in the accurate and efficient interpretation of the images. Examples of pathology stains which can be reproduced using the methods of the present invention include, but are not limited to, hematoxylin, eosin, wright, giemsa, Masson's trichrome, Jones, trichrome, periodic acid Schiff (PAS) and reticulin stains. Combinations of pathology stains can also be reproduced using methods of the present invention. In one embodiment, the combination of pathology stains is hematoxylin and eosin (H&E). In another embodiment, the combination of pathology stains is wright and giemsa.

In some embodiments, the step of imaging the sample is performed in combination with an additional imaging method, resulting in multi-modal imaging. In one embodiment, the additional imaging method is a higher-order harmonic generation. Higher-order harmonic generation permits the recreation of additional specialized histological stains, such as collagen stains like trichrome and silver stains like Jones stain. In one embodiment, the higher order harmonic generation is second harmonic generation (SHG). SHG results from multiphoton excitation of asymmetric repeating proteins such as collagen, and may be used for simple identification and quantification of collagen fibrosis and amyloid in combination with the imaging method, such as MPM. In one embodiment, the additional imaging method is Fluorescence Lifetime Imaging. Fluorescence Lifetime Imaging may be used to provide additional contrast in MPM by distinguishing between fluorophores with differing lifetime characteristics, or by distinguishing between fluorescence and SHG. In another embodiment, the additional imaging method uses multiple fluorescent antibodies. Multiple fluorescent antibodies may be used to provide potential for performing immunohistochemistry in uncut samples with multiple antigens detectable on the same cells. In another embodiment, the imaging method is used in combination with diode lasers. See Dechet et al., 2003, J. Urol. 169:71-74 and Durfee et al., 2012, Opt. Express 20:13677-13683, each which is incorporated by reference herein in its entirety. Other techniques known in the art to increase the rate of scanning of the sample image may be used in the imaging step, as would be understood by one of ordinary skill in the art. Non-limiting examples include multibeam scanning systems, spatiotemporal multiplexing, and temporal focusing. See Bewersdorf et al., 1998, Opt. Lett. 23:655-657, Amir et al., 2007, Opt. Lett. 32-1731-1733, Oron et al., 2005, Opt. Express 13:1468-1476, and Zhu et al., 2005, Opt. Express 13:2153-2159, each which is incorporated by reference herein in its entirety. In another embodiment, the imaging step is performed in real time using video imaging.

The methods of the present invention provide a clear, high-quality image of the sample obtained at a greater sample depth as compared with more traditional histological methods, such as sample treated only with formalin fixation. In one embodiment, the sample image is obtained at a sample depth of about 100 nm to about 2 cm. In another embodiment, the sample image is obtained at a sample depth of about 100 nm to about 500 μm. In another embodiment, the sample image is obtained at a sample depth of about 100 nm to about 1 cm. In another embodiment, the sample image is obtained at a sample depth of about 50 μm to about 500 μm. In another embodiment, the sample image is obtained at a sample depth of about 100 nm to about 100 μm. In another embodiment, the sample image is obtained at a sample depth of about 200 μm. In another embodiment, the sample image is obtained at a sample depth of about 100 μm.

In one embodiment, imaging of the sample provides digital sample data. This digital data may then be stored for later distribution, such as for consultation and health records, thereby improving the accessibility of the images for further evaluation or reevaluation. In addition, digital sample data is capable of maintaining the integrity of the data, as opposed to physical samples which may be lost or damaged and cannot be stored digitally.

In one embodiment, the sample image is a three-dimensional (3-D) sample image. A 3-D sample image can be produced using any method known in the art, as would be understood by one skilled in the art. In one embodiment, the 3-D sample image is produced from an entire biopsy. In another embodiment, a 3-D sample image produced from a whole biopsy provides a quantitative approach to diagnosing a disease. In another embodiment, a 3-D sample image of the present invention provides facile identification of subtle morphologic findings in the imaging sample. For example, 3-D sample images improve the quantitative and qualitative analysis of fibrosis observed in various conditions such as cirrhosis, hypertensive renal disease, interstitial lung disease, and ovarian cancer over other two-dimensional (2-D) histological methods currently known in the art. In another embodiment, a 3-D sample image of the present invention is used to diagnose a malignant growth. In one embodiment, the methods of the present invention provide full rotational control of 3-D sample images. Diagnosis of malignant growth is often dependent on visualizing growth patterns, particularly in glandular-based disorders such as prostate and breast cancer. Such analysis has been primarily based on the two-dimensional orientation, which may require pathologists to look back-and-forth at (hopefully) contiguous segments in order to render a diagnosis. In these 2D methods, visual inspection can be further complicated by poor embedding and orientation differences of the sample. In non-limiting examples, the 3-D sample images of the present invention are used to diagnose metastatic colon cancer in liver and for the diagnosis of endometrial abnormalities. In another non-limiting example, 3-D reconstructions of MPM imaging from clarified tissue may be used on complete biopsy-sized tissue specimens and may also be used to produce quantifiable characterization of collagen fibrosis. Other non-limiting examples of the use of 3-D sample images include identification of low-grade abnormalities in glandular cell growth, such as with prostate and breast neoplasia, the evaluation of depth of invasion of tumors, such as for determining depth of muscle invasion in bladder biopsies, and the more complete quantitative evaluation of fibrosis, of particular significance in kidney and liver biopsies.

### Microscope System

In another aspect, the invention relates to a microscope system for 2-D and 3-D imaging of specimens. In one embodiment, the microscope system is amenable for use in the methods of the present invention as described elsewhere herein.

An exemplary microscope system may comprise a laser, an optional beam shaper, spinning polygon, scan lens, tube lens, microscope objective, dichroic mirror A, dichroic mirror B, a plurality of emission filters, a plurality of detectors, and translation stage. A microscope system of the invention may further comprise any component customarily used in other similar microscope systems, such as viewfinders, power sources, stage manipulation mechanisms, autofocus mechanisms, automation systems, and the like.

The laser comprises a laser source providing, for example, multiphoton excitation of dyes used in sample labeling. The laser may be any suitable laser, such as femtosecond or picosecond pulsed fiber laser, or any other short-pulse laser with a center wavelength that is adjustable for optimum excitation of dyes. Alternatively, the center wavelength may be non-tunable but may be chosen to correspond to a wavelength suitable for exciting dyes used to stain a sample. In one embodiment, the center wavelength is 780 nm.

In one embodiment, a laser beam being emitted by the laser passes through beam shaper. The beam shaper expands the laser beam to a width required to illuminate the back aperture of microscope objective. In another embodiment, the beam shaper may transform the laser beam profile. For example, a laser beam having a Gaussian beam profile may be transformed to have a flat top profile after passing through the beam shaper, which results in more efficient illumination of the back aperture of microscope objective in other embodiments, a beam shaper is not used.

A laser beam is rapidly scanned in angle by a spinning polygon having a plurality of mirrored facets. In various alternative embodiments, the laser beam may be rapidly scanned in angle by any one of several resonant scanning mechanisms commonly available for rapid beam scanning. Non-limiting examples of resonant scanning mechanisms used for rapid beam scanning include resonant galvanometers and digital micromirror devices.

A rapidly scanned laser beam is imaged to the back aperture of the microscope objective using a scan lens and tube lens 40. In one embodiment, the scan lens is a telecentric lens. the scan lens 38 can be an F-theta lens. In various embodiments, the scan lens is any suitable lens that images an angle-scanned beam from the surface of the spinning polygon to the back aperture of microscope objective when used in conjunction with tube lens.

In one embodiment, the microscope system comprises a dichroic mirror, such that fluorescent light collected by the objective lens may be reflected by the dichroic mirror to be collected by one or more detectors. Alternatively, the beam path may be chosen such that dichroic mirror is used to reflect the excitation laser beam to the back aperture of a microscope objective to transmit the collected fluorescence to the detectors. In one embodiment, fluorescence or SHG may be collected as transmitted light with or without a collecting lens (not pictured) and detectors. Detectors may be any detector sensitive to appropriate wavelengths. For example, in one embodiment the detectors comprise photomultiplier tubes. The system may further comprise a dichroic mirror, or any number of additional dichroic mirrors as is needed, to send different color fluorescence to different detectors. In one embodiment, the system may further comprise any number of emission filters as is needed, wherein the emission filters placed in front of the detectors reject stray laser light and other light interference.

In various embodiments, a microscope system of the invention comprises at least one microscope objective. In one embodiment, the microscope objective is compatible with a high-refractive-index immersion medium. In various embodiments, the objective is compatible with a high-refractive-index immersion medium having an index between 1.45 and 1.6. In one embodiment, the microscope objective is compatible with a high-refractive-index immersion medium having an index of 1.54. In another embodiment, the microscope objective is compatible with an immersion medium that is the BABB clearing agent as disclosed elsewhere herein. In alternative, the microscope system has an inverted microscope arrangement comprising a microscope objective with a relatively broad and flat aspect on the side that faces a sample such that immersion fluid can be easily maintained between the objective and a sample cartridge.

The microscope objective may comprise a numerical aperture for high quality optical axial sectioning with a short depth-of-field. In one embodiment, the numerical aperture is at least 0.8. In another embodiment, the numerical aperture is at least 1.0. Alternatively, the microscope objective 41 comprises a large field of view for imaging a sample. In one embodiment, the objective has a field of view that is approximately 500 microns. In another embodiment, the microscope objective has a field of view that is greater than approximately 500 microns.

Samples may be placed on a translation stage for imaging. The position of the translation stage relative to microscope objective may be adjusted to any position for imaging different regions of samples and for adjusting focus. For example, in various embodiments, the translation stage may be adjusted such that the working distance between the microscope objective and a sample is between 100 μm and 2 mm. In one embodiment, the working distance may be adjusted to be at least 100 μm to achieve sufficient depth such that complete optical sections may be obtained in samples with significant peaks and valleys from surface irregularities.

### Methods of Using the Microscope System

In another aspect, the invention relates to methods of imaging samples using the microscope system of the present invention. The method passes a laser beam through the system such that a microscope objective focuses the laser beam upon a single point on a sample placed on a translation stage. In some embodiments, a spinning polygon causes the laser beam to rapidly scan a line across the sample. In other embodiments, a resonant galvanometer directs the laser beam to rapidly scan a line across the sample.

In one embodiment, the laser beam is rapidly scanned across the sample in a stepped linear fashion (raster scan), such that successive scans produces a rectangular 2-D image. In a further embodiment, the translation stage may be moved along the imaging plane after a rectangular 2-D image is obtained such that a second rectangular 2-D image may be obtained by rapidly scanning the laser beam across the sample in a stepped linear fashion. The translation stage may be moved along the imaging plane in further incremental steps such that a plurality of rectangular 2-D images are obtained, until a full cross-section of the specimen at a given imaging plane has been interrogated. The plurality of rectangular 2-D imagines are stitched together using any method known in the art, such as with microscopy software, to create a full cross-section image of the sample. In one embodiment, 2-D images having partial overlap aids the automatic assembly of the cross-section of the sample.

In further embodiments, the specimen may be translated axially relative to the objective so that the process of obtaining 2-D images at a given image plane may be repeated, representing a different cross sectional image of the sample. In one embodiment, successive rectangular 2-D image cross-sections are obtained until the full volume of the sample has been interrogated.

### Kits of the Invention

The invention also includes a kit comprising components useful within the methods of the invention and instructional material that describes, for instance, the method of processing tissue samples as described elsewhere herein. The kit may comprise reagents useful for performing the methods of the invention. For instance, the kit may comprise reagents such as fixatives, dyes, and clearing solutions. The kit may further comprise devices useful for performing the methods of the invention. For instance, the kit may comprise the specimen holding device and the microscope system of the invention, as described elsewhere herein.

In one embodiment, the reagents are provided in concentrated form, such that the weight and size of the kit can be reduced and the solutions need only be diluted for immediate use. In another embodiment, the kit further comprises (preferably sterile) the components of the reagents in lyophilized form. For instance, the components may be in premeasured amounts suitable for reconstitution and immediate use. The kit can further include one or more additional component, such as reconstitution containers, and additional reagents such as deionized water, wash buffer, and the like.

In certain embodiments, the kit comprises instructional material. Instructional material may include a publication, a recording, a diagram, or any other medium of expression which can be used to communicate the usefulness of the device or kit described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet.

High concentrations of certain protein dyes in protein dense tissues have been found to result in markedly increased absorption of fluorescent signal from deeper levels, thereby decreasing contrast and degrading image quality. That phenomenon can be observed when using any method of optical sectioning microscopy such as confocal imaging, multiphoton imaging, and selective plane illumination and supports the counterintuitive inverse relationship underpinning certain methods of the invention.

Depending on the relative emission and absorption spectra of the dyes employed, the increased absorption described above can affect both nuclear and protein-specific fluorescent dye signals. However, the relative concentration of protein in tissue is much higher than nucleic acids and, accordingly, protein content and protein-specific dye concentration are the significant drivers in signal weakening in a combined protein and nucleic acid staining assay. The inverse dye concentration to protein content methods described herein can be applied to both single-stain assays as well as combined protein and nucleic acid stain assays. Inversely varying protein-specific stain concentration based on sample protein content benefits contrast and signal intensity not only in protein dye imaging but also in imaging of nuclear dyes in the aforementioned combined stain assays.

Using a fluorescent protein dye concentration inversely related to protein density of the tissue sample also provides the benefit of reducing waste and, therefore reagent costs by conserving dye when applied to imaging at any depth. However, the signal weakening at depth associated with saturation from a too-high dye to protein content ratio can be observed when imaging beyond about 200 μm in protein-rich tissue. Accordingly, additional image quality benefits can be observed in optical sectioning microscopy techniques that rely on imaging at such depths.

There may be a minimum and maximum level of dye concentration that can be applied regardless of protein concentration. At very low concentrations of protein there is a threshold level of dye concentration above which the protein is saturated and any additional stain is wasted. At very high protein densities, there is a minimum threshold of dye concentration required to achieve any dye penetration and fluorescent signal. It may, therefore, be in the middle range of tissue protein densities where variations in dye concentration inverse to protein density yield maximum benefit.

As detailed below, methods of the invention are particularly applicable when processing and imaging tissue samples with relatively high (e.g., epithelial tissue) or low (e.g., renal tissue) protein content or in tissue where fibrosis is a diagnostic indicator (e.g., kidney or liver tissue). The table below notes examples of tissues and average protein content.

| Tissue | Approximate % by mass protein |
|---|---|
| Adipose | 4 |
| Connective Tissue | 37 |
| Brain Tissue | 10 |
| Heart Tissue | 17 |
| Lung | 17 |
| Liver | 18 |
| Kidney | 18 |
| Skeletal Muscle | 20 |
| Pancreatic Tissue | 13 |
| Cartilage | 11 |
| Yellow Marrow | 4 |
| Red Marrow | 20 |
| Bone Tissue | 22 |
| Skin | 25 |
| Ovary Tissue | 14 |
| Thyroid Tissue | 14 |

While overall protein content can help inform decisions on dye concentration, protein density is of primary importance. For example, as noted in the examples, kidney tissue is composed of tubular structures and is highly vascularized leading to a lower protein density than might be indicated by its level of overall protein content. As discussed herein, deep tissue imaging of kidney tissue accordingly tolerates a higher dye concentration than a protein-dense tissue such as epithelium.

### EXAMPLES

Exemplary applications of the inventive methods include H&E-like images at depth of kidney and skin, obtained by multiphoton laser excitation after simultaneous fluorescent staining with the protein-specific fluorescent dye eosin and the nucleic acid-specific fluorescent dye DAPI. Although both tissue types might seem superficially to represent similarly dense tissues, they differ significantly in overall protein concentration. Kidneys are composed of tubular structures and are highly vascular, resulting in significantly lower overall protein content than the skin epithelium, which is composed of tightly packed cells with relatively few vessels and usually possess an overlying layer of tightly packed keratin protein without vascularization. Relatively high concentration eosin dye in fluorescent kidney protein staining, such as about of alcoholic stock solution, results in a clear visualization of diagnostically relevant structures such as capillary walls of the blood filtering unit called the glomerulus, and images collected over a depth of about 1 mm are essentially unaffected by the protein dye that lies between the excited fluorophore and the fluorescence detectors. The same high concentration of protein fluorescent dyes results in marked dimming of both the eosin and DAPI signals in images beyond about 200 μm deep in relatively protein-rich keratinized epithelial tissue. However, performing the same dyeing procedure as above but with a 10-fold lower concentration of eosin (about 0.04% of alcoholic stock solution) resulted in adequate contrast of protein structures at surface as well as more homogeneously bright and readable staining at depths beyond 300 μm. As such, varying concentrations of the fluorescent dye for protein staining are preferred based on the overall protein content of the specimen.

A second exemplary application is in the three-dimensional characterization of collagen in liver. Although liver is not an epithelial tissue, it has a relatively high protein content which is related to its high metabolic activity as a primary detoxifying organ. Collagen has a property that is useful for imaging using multiphoton pulsed laser excitation. If the intensity of light pulses is high enough, such as when a femtosecond pulsed laser is focused by a microscope objective, then the energy of two photons reaching the collagen fiber simultaneously is combined into one photon of twice the energy (half the wavelength). In liver, that second harmonic generation (SHG) signal is derived almost exclusively from collagen and can thus be exploited as a quantifiable measure of fibrosis. Fibrosis is a marker of liver injury in a variety of hepatic conditions and an important clinical prognostic factor. Visualization of collagen throughout a liver specimen is particularly desirable as it represents a more extensive and thereby precise measure of sample collagen. Accordingly, the application of the inventive methods to provide detailed SHG analysis at depth in hepatic tissue has notable diagnostic and prognostic benefits.

Normalization of the refractive index throughout a tissue specimen, such as through clearing, can further improve collagen maps of thick liver and other tissue specimens. SHG analysis can be performed concurrently with protein and nucleic acid imaging, reducing overall work and processing time while maximizing informational content derived with imaging. However, in such combined applications, while using wavelengths between about 750 nm and 900 nm, the SHG signal is severely attenuated at depth if the concentration of eosin, staining the protein, is high. When the SHG signal is collected in transmittance rather than through the excitation lens, the effect is such that surface excitation of SHG at higher protein dye concentrations results in a more attenuated signal. In both scenarios, minimizing eosin concentration during staining to a degree that still allows visualization of microscopic protein-rich structures aids in maximizing the depth at which collagen signal can be simultaneously detected via SHG analysis.

For liver specimens, using staining methods, processing reagents, and multiphoton imaging systems as have been previously described, eosin concentrations around 0.02% of alcoholic stock solutions were found to yield an appropriate balance of protein signal for adequate reproduction of H&E stains while permitting imaging of collagen SHG at several hundred micrometers.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of processing a tissue sample, the method comprising:
obtaining a tissue sample; and
contacting the tissue sample with a solution comprising methanol, a fluorescent nuclear dye at a concentration between about 300 μM and about 800 nM, and a fluorescent protein dye at a concentration between about 0.025 nM and about 2.5 nM, the solution having a pKa between about 4.84 and about 5.

2. The method of claim 1, further comprising clearing the tissue, sample with a clearing solution having a refractive index between about 1.5 to about 1.57.

3. The method of claim 1, further comprising incubating the tissue sample in formalin before the contacting step.

4. The method of claim 3, wherein the tissue sample is incubated in formalin for at least 5 minutes.

5. The method of claim 1, further comprising producing a tissue sample image by measuring fluorescence of the tissue sample after the contacting step.

6. The method of claim 5, wherein the tissue sample is not exposed to chloroform before the producing step.

7. The method of claim 1, wherein the fluorescent nuclear dye comprises 2-(4-amidinophenyl)-1H-indole-6-carboxamidine (DAPI).

8. The method of claim 1, wherein the fluorescent nuclear dye comprises Hoechst dye.

9. The method of claim 1, wherein the solution further comprises acetic acid.

10. The method of claim 1, wherein the clearing agent comprises benzyl alcohol/benzyl benzoate in a 1:2 ratio.

11. The method of claim 1, wherein the contacting step is performed at a temperature between about 40° C. and about 47° C.

12. The method of claim 3, wherein the incubating step is performed at a temperature between about 40° C. and about 47° C.

13. The method of claim 1, further comprising storing the solution at about between about −20° C. before the contacting step.

14. The method of claim 1, wherein the fluorescent protein dye comprises eosin.

15. A method of imaging a tissue sample, the method comprising:
clearing an undyed tissue sample with a clearing agent;
exposing the cleared, undyed tissue sample with pulsed laser light to produce an image of second harmonic generating (SHG) molecules indicative of collagen content and distribution within the undyed tissue sample;
contacting the cleared, undyed tissue sample with alcohol to remove the clearing agent;
dyeing the undyed tissue sample with one or more fluorescent dyes in an alcohol solution;
clearing the dyed tissue sample;
imaging the cleared, dyed tissue sample to produce an image of fluorescence signal indicative of content and distribution of dyed molecules within the dyed tissue sample.

16. The method of claim 15, wherein imaging the cleared, dyed tissue is performed with an optical sectioning microscope to obtain one or more levels of fluorescence distribution and intensity information.

17. The method of claim 15, wherein the clearing agent has a refractive index of about 1.47 or more.

18. The method of claim 15, wherein the undyed tissue sample is fixed before the clearing step.

19. The method of claim 18, wherein the undyed tissue sample is fixed in formalin.

20. The method of claim 15, wherein the pulsed laser light comprises a wavelength of about 780 nm.

21. The method of claim 15, wherein the cleared, undyed tissue sample is exposed to the pulsed laser light and the image of second harmonic generating (SHG) molecules is produced through a single objective lens.

22. The method of claim 15, wherein the clearing step comprises:
incubating the undyed tissue sample in methanol for sufficient time to substantially replace water in the specimen with methanol; and
submerging the incubated, undyed tissue sample in the clearing agent, wherein the clearing agent comprises a high refractive index clearing fluid.

23. The method of claim 22, wherein the high refractive index clearing fluid comprises benzyl alcohol/benzyl benzoate in a 1:2 ratio.

24. The method of claim 22, wherein the high refractive index clearing fluid comprises ethyl cinnamate.

25. The method of claim 22, wherein the incubation step is between about 5 minutes and about 8 hours.

26. The method of claim 22, wherein the submerging step is between about 5 minutes and about 1 hour.

27. The method of claim 15, wherein the one or more fluorescent dyes comprise a fluorescent nuclear dye and a fluorescent protein dye.

28. The method of claim 27, wherein the fluorescent nuclear dye comprises 2-(4-amidinophenyl)-1H-indole-6-carboxamidine (DAPI).

29. The method of claim 27, wherein the fluorescent protein dye comprises eosin.

30. The method of claim 15, wherein the alcohol solution comprises methanol and acetic acid.

31. The method of claim 15, wherein the dying and clearing steps are performed at between about 37° C. and about 50° C.

32. The method of claim 16, wherein the optical sectioning microscope is selected from the group consisting of a confocal microscope, selective plane illumination microscope, deconvolution microscope, and a multiphoton microscope.

33. A method of non-microscopic imaging, the method comprising the steps of substantially immobilizing a biological sample;
obtaining an image of the sample;
storing the image in a database along with non-image-based identifying information; and
recalling the non-image-based information from the database and matching a newly acquired non-microscopic image of the biological sample to the image stored into the database.

34. The method of claim 33, wherein the image is obtained using a camera.

35. The method of claim 33, wherein the image is stored in the database as a fluorescence image.

36. The method of claim 33, wherein the non-image-based identifying information is selected from a numerical identifier, an optical bar code, a patient name, and a collector's name.

37. The method of claim 33, wherein the newly acquired non-microscopic image is of the same type as the images stored in the database.

38. The method of claim 33, wherein the newly acquired non-microscopic image is of a different type as the images stored in the database.

39. The method of claim 33, wherein the image of the sample is preprocessed to adjust attributes selected from brightness, contrast, and color balance.

40. The method of claim 33, wherein the biological sample is a needle core biopsy, a surgical resection, or a slice of tissue mounted on a microscope slide.

41. The method of claim 33, wherein the newly acquired image or images are matched to images stored in the database by use of image cross-correlation.
